(19) United States
(12) Patent Application Publication
(10) Pub. No.: US 2022/0332843 A1
(43) Pub. Date: Oct. 20, 2022

(54) ANTIBODIES AND METHOD OF PRODUCING ANTIBODIES

(71) Applicant: ARIZONA BOARD OF REGENTS ON BEHALF OF ARIZONA STATE UNIVERSITY, Scottsdale, AZ (US)

(72) Inventors: Stephen Albert JOHNSTON, Scottsdale, AZ (US); Milene PETERSON, Scottsdale, AZ (US)

(21) Appl. No.: 17/808,402

(22) Filed: Jun. 23, 2022

Related U.S. Application Data
(63) Continuation of application No. PCT/US2021/012335, filed on Jan. 6, 2021.
(60) Provisional application No. 62/959,067, filed on Jan. 9, 2020.

(51) Int. Cl. C07K 16/30; C12N 15/10; A61P 35/00

(52) U.S. Cl. CPC C07K 16/30; C12N 15/1037; A61P 35/00; C07K 2317/10; C07K 2317/73

(57) ABSTRACT

A method of producing a therapeutic or preventive antibody for a cancer and/or tumor of interest is disclosed. The methods include binding antibodies of interest to frameshift peptide arrays, selecting frameshift peptides that are immunoreactive to the antibodies, and preparing an antibody composition against the selected frameshift peptides. Also disclosed are antibody compositions, methods of eliciting an immune response and methods of treatment using the methods of producing the antibodies as described herein.

### CROSS-REFERENCE TO RELATED APPLICATIONS

This application is a continuation application of PCT International Application No. PCT/US2021/012335, filed on Jan. 6, 2021, and is based on, and claims a priority benefit from U.S. Provisional Patent Application No. 62/959,067, filed Jan. 9, 2020, each of which is incorporated herein by reference in its entirety.

### ACKNOWLEDGMENT OF GOVERNMENT SUPPORT

This invention was made with government support under R21 CA220150 awarded by the National Institutes of Health. The government has certain rights in the invention.

### FIELD OF THE DISCLOSURE

This application concerns antibodies and, more specifically, methods of producing antibodies, such as therapeutic antibodies against, tumor-specific frameshift neoantigens.

### BACKGROUND

Herceptin was a revolution for the cancer field. It was discovered that some breast cancers overproduce the Her2 protein and present it on the surface of the tumor. Antibodies (Herceptin) that bind to the protein could kill the tumor cells. Since then several other forms of monoclonal and bi-specific antibody therapeutics have been developed to treat cancer. These binding agents can work directly to kill tumor cells, by modifying the tumor cell's activity or indirectly by delivering a toxin or a radioactive material to the tumor. A major challenge with these approaches is to find a target on the tumor cell that is common among enough patients (in order to justify production costs) and also not on normal cells. Another challenge is that the tumor can escape the therapeutic by eliminating production of the target. For example, the tumor can stop making Her2 protein by multiple mechanisms or mutate the protein so it is not recognized by the antibody.

It is of note that the breast cancer cells overproducing Her2 protein often elicit antibodies to the protein. Yet, the tumor progresses. Apparently, the amount of antibody produced naturally to the tumor is not enough to kill it. The dose of Herceptin (8 mg/kg) would be more 100× than produced naturally. Therefore, additional therapies are needed to address the limitations associated with the current therapies. What is needed to advance therapy for cancer are therapeutic antibodies that 1) can be used against many cancers, therefore reducing costs, 2) target proteins that cannot be eliminated by mutations, and 3) are against tumor specific targets so they can be safely used against early cancers.

### SUMMARY

Disclosed herein are methods of producing antibodies, antibodies produced therefrom and methods of treatment that meet the requirements mentioned above. In some embodiments, a methods of producing antibodies include providing one or more biological samples obtained from one or more subjects identified as having the cancer or tumor of interest to a substrate comprising frameshift peptides produced by one or more tumors; detecting antibodies which bind to the frameshift peptides; selecting the frameshift peptides that are immunoreactive with the antibodies; and preparing an antibody composition against the selected frameshift peptides for the cancer or tumor of interest. This approach is facilitated by the discovery that cancer cells frequently and recurrently across individual cancers make errors on RNA processing that create frameshift neoantigens. Therefore, it is possible to make antibody therapeutics against these frameshift peptides that can be used off-the-shelf to treat cancers.

In some embodiments, the one or more biological samples comprises a set of biological samples obtained from the set test subjects using antibody reactivity. In some embodiments, antibody reactivity is detected using an antibody assay comprises ELISA, radioimmunoassay, western blot, surface plasmon resonance, immunostaining, immunoprecipitation, mass spectrometry, phage display, ELISPOT, flow cytometry, cytometric bead array, immunohistochemistry, high density array, microarray, delayed-type hypersensitivity (DTH), and combinations thereof. In some embodiments, the frameshift peptides comprises peptides encoded by a frameshifted mRNA expressed by a cancer cell. In some embodiments, the frameshifted mRNA is created in a splicing error, a transcription insertion or deletion error, or a mis-initiation of translation error. In some embodiments, the frameshift peptides binds to at least one MHC subtype. In some embodiments, the frameshift peptides comprises at least one T cell epitope. In some embodiments, the frameshift peptides comprises at least on B cell epitope. In some embodiments, the frameshift peptides are presented on the cancer cell surface. In some embodiments, the frameshift peptides are part of an array or a phage display library. In some embodiments, the antibodies to frameshift peptides are screened for binding to and/or killing tumor cells. In some embodiments, the one or more biological samples comprises one or more of blood, plasma, serum, thymus, bone marrow, spleen, lymph node, bronchoalveolar lavage, breast, central nervous system, cerebrospinal fluid, eye, tears, gastrointestinal tract, saliva, feces, urine, heart, kidney, liver, lung, muscle, pancreas, peripheral nervous system, saliva, skin, thyroid, trachea, and tumor. In some embodiments, the antibody composition further comprises a pharmaceutically acceptable adjuvant or excipient. In some embodiments, the antibody composition further comprises an immune checkpoint inhibitor. In some embodiments, the methods further comprises administering an effective amount of the antibody composition to a subject in need thereof to treat the cancer and/or tumor.

In some embodiments, treating the cancer and/or tumor comprises reducing tumor size, inhibiting tumor growth, reducing tumor burden, increasing survival, or increasing cancer-free survival. In some embodiments, administering the antibody composition elicits an immune response in the subject against the cancer.

In some embodiments, the cancer is selected from the group consisting of Acanthoma, Acinic cell carcinoma, Acoustic neuroma, Acral lentiginous melanoma, Acrospiroma, Acute eosinophilic leukemia, Acute lymphoblastic leukemia, Acute megakaryoblastic leukemia, Acute monocytic leukemia, Acute myeloblastic leukemia with maturation, Acute myeloid dendritic cell leukemia, Acute myeloid leukemia, Acute promyelocytic leukemia, Adamantinoma, Adenocarcinoma, Adenoid cystic carcinoma, Adenoma, Adenomatoid odontogenic tumor, Adrenocortical carcinoma, Adult T-cell leukemia, Aggressive NK-cell leukemia, AIDS-Related Cancers, AIDS-related lymphoma, Alveolar soft part sarcoma, Ameloblastic fibroma, Anal cancer, Anaplastic large cell lymphoma, Anaplastic thyroid cancer, Angioimmunoblastic T-cell lymphoma, Angiomyolipoma, Angiosarcoma, Appendix cancer, Astrocytoma, Atypical teratoid rhabdoid tumor, Basal cell carcinoma, Basal-like carcinoma, B-cell leukemia, B-cell lymphoma, Bellini duct carcinoma, Biliary tract cancer, Bladder cancer, Blastoma, Bone Cancer, Bone tumor, Brain Stem Glioma, Brain Tumor, Breast Cancer, Brenner tumor, Bronchial Tumor, Bronchioloalveolar carcinoma, Brown tumor, Burkitt' s lymphoma, Cancer of Unknown Primary Site, Carcinoid Tumor, Carcinoma, Carcinoma in situ, Carcinoma of the penis, Carcinoma of Unknown Primary Site, Carcinosarcoma, Castleman's Disease, Central Nervous System Embryonal Tumor, Cerebellar Astrocytoma, Cerebral Astrocytoma, Cervical Cancer, Cholangiocarcinoma, Chondroma, Chondrosarcoma, Chordoma, Choriocarcinoma, Choroid plexus papilloma, Chronic Lymphocytic Leukemia, Chronic monocytic leukemia, Chronic myelogenous leukemia, Chronic Myeloproliferative Disorder, Chronic neutrophilic leukemia, Clear-cell tumor, Colon Cancer, Colorectal cancer, Craniopharyngioma, Cutaneous T-cell lymphoma, Degos disease, Dermatofibrosarcoma protuberans, Dermoid cyst, Desmoplastic small round cell tumor, Diffuse large B cell lymphoma, Dysembryoplastic neuroepithelial tumor, Embryonal carcinoma, Endodermal sinus tumor, Endometrial cancer, Endometrial Uterine Cancer, Endometrioid tumor, Enteropathy-associated T-cell lymphoma, Ependymoblastoma, Ependymoma, Epithelioid sarcoma, Erythroleukemia, Esophageal cancer, Esthesioneuroblastoma, Ewing Family of Tumor, Ewing Family Sarcoma, Ewing's sarcoma, Extracranial Germ Cell Tumor, Extragonadal Germ Cell Tumor, Extrahepatic Bile Duct Cancer, Extramammary Paget's disease, Fallopian tube cancer, Fetus in fetu, Fibroma, Fibrosarcoma, Follicular lymphoma, Follicular thyroid cancer, Gallbladder Cancer, Gallbladder cancer, Ganglioglioma, Ganglioneuroma, Gastric Cancer, Gastric lymphoma, Gastrointestinal cancer, Gastrointestinal Carcinoid Tumor, Gastrointestinal Stromal Tumor, Gastrointestinal stromal tumor, Germ cell tumor, Germinoma, Gestational choriocarcinoma, Gestational Trophoblastic Tumor, Giant cell tumor of bone, Glioblastoma multiforme, Glioma, Gliomatosis cerebri, Glomus tumor, Glucagonoma, Gonadoblastoma, Granulosa cell tumor, Hairy Cell Leukemia, Hairy cell leukemia, Head and Neck Cancer, Head and neck cancer, Heart cancer, Hemangioblastoma, Hemangiopericytoma, Hemangiosarcoma, Hematological malignancy, Hepatocellular carcinoma, Hepatosplenic T-cell lymphoma, Hereditary breast-ovarian cancer syndrome, Hodgkin Lymphoma, Hodgkin's lymphoma, Hypopharyngeal Cancer, Hypothalamic Glioma, Inflammatory breast cancer, Intraocular Melanoma, Islet cell carcinoma, Islet Cell Tumor, Juvenile myelomonocytic leukemia, Kaposi Sarcoma, Kaposi's sarcoma, Kidney Cancer, Klatskin tumor, Krukenberg tumor, Laryngeal Cancer, Laryngeal cancer, Lentigo maligna melanoma, Leukemia, Lip and Oral Cavity Cancer, Liposarcoma, Lung cancer, Luteoma, Lymphangioma, Lymphangiosarcoma, Lymphoepithelioma, Lymphoid leukemia, Lymphoma, Macroglobulinemia, Malignant Fibrous Histiocytoma, Malignant fibrous histiocytoma, Malignant Fibrous Histiocytoma of Bone, Malignant Glioma, Malignant Mesothelioma, Malignant peripheral nerve sheath tumor, Malignant rhabdoid tumor, Malignant triton tumor, MALT lymphoma, Mantle cell lymphoma, Mast cell leukemia, Mediastinal germ cell tumor, Mediastinal tumor, Medullary thyroid cancer, Medulloblastoma, Medulloepithelioma, Melanoma, Meningioma, Merkel Cell Carcinoma, Mesothelioma, Metastatic Squamous Neck Cancer with Occult Primary, Metastatic urothelial carcinoma, Mixed Mullerian tumor, Monocytic leukemia, Mouth Cancer, Mucinous tumor, Multiple Endocrine Neoplasia Syndrome, Multiple myeloma, Mycosis Fungoides, Myelodysplastic Disease, Myelodysplastic Syndromes, Myeloid leukemia, Myeloid sarcoma, Myeloproliferative Disease, Myxoma, Nasal Cavity Cancer, Nasopharyngeal Cancer, Nasopharyngeal carcinoma, Neoplasm, Neurinoma, Neuroblastoma, Neurofibroma, Neuroma, Nodular melanoma, Non-Hodgkin lymphoma, Nonmelanoma Skin Cancer, Non-Small Cell Lung Cancer, Ocular oncology, Oligoastrocytoma, Oligodendroglioma, Oncocytoma, Optic nerve sheath meningioma, Oral cancer, Oropharyngeal Cancer, Osteosarcoma, Ovarian cancer, Ovarian Epithelial Cancer, Ovarian Germ Cell Tumor, Ovarian Low Malignant Potential Tumor, Paget's disease of the breast, Pancoast tumor, Pancreatic cancer, Papillary thyroid cancer, Papillomatosis, Paraganglioma, Paranasal Sinus Cancer, Parathyroid Cancer, Penile Cancer, Perivascular epithelioid cell tumor, Pharyngeal Cancer, Pheochromocytoma, Pineal Parenchymal Tumor of Intermediate Differentiation, Pineoblastoma, Pituicytoma, Pituitary adenoma, Pituitary tumor, Plasma Cell Neoplasm, Pleuropulmonary blastoma, Polyembryoma, Precursor T-lymphoblastic lymphoma, Primary central nervous system lymphoma, Primary effusion lymphoma, Primary Hepatocellular Cancer, Primary Liver Cancer, Primary peritoneal cancer, Primitive neuroectodermal tumor, Prostate cancer, Pseudomyxoma peritonei, Rectal Cancer, Renal cell carcinoma, Respiratory Tract Carcinoma Involving the NUT Gene on Chromosome 15, Retinoblastoma, Rhabdomyoma, Rhabdomyosarcoma, Richter's transformation, Sacrococcygeal teratoma, Salivary Gland Cancer, Sarcoma, Schwannomatosis, Sebaceous gland carcinoma, Secondary neoplasm, Seminoma, Serous tumor, Sertoli-Leydig cell tumor, Sex cord-stromal tumor, Sezary Syndrome, Signet ring cell carcinoma, Skin Cancer, Small blue round cell tumor, Small cell carcinoma, Small Cell Lung Cancer, Small cell lymphoma, Small intestine cancer, Soft tissue sarcoma, Somatostatinoma, Soot wart, Spinal Cord Tumor, Spinal tumor, Splenic marginal zone lymphoma, Squamous cell carcinoma, Stomach cancer, Superficial spreading melanoma, Supratentorial Primitive Neuroectodermal Tumor, Surface epithelial-stromal tumor, Synovial sarcoma, T-cell acute lymphoblastic leukemia, T-cell large granular lymphocyte leukemia, T-cell leukemia, T-cell lymphoma, T-cell prolymphocytic leukemia, Teratoma, Terminal lymphatic cancer, Testicular cancer, Thecoma, Throat Cancer, Thymic Carcinoma, Thymoma, Thyroid cancer, Transitional Cell Cancer of Renal Pelvis and Ureter, Transitional cell carcinoma, Urachal cancer, Urethral cancer, Urogenital neoplasm, Uterine sarcoma, Uveal melanoma, Vaginal Cancer, Verner Morrison syndrome, Verrucous carcinoma, Visual Pathway Glioma, Vulvar Cancer, Waldenstrom's macroglobulinemia, Warthin's tumor, or Wilms' tumor.

Some embodiments provided herein relate to antibody compositions produced by the methods provided herein.

Some embodiments provided herein relate to methods of eliciting an immune response in a treatment subject in need thereof. In some embodiments, the methods include selecting a treatment subject with cancer or at risk for developing cancer; and administering a disclosed antibody composition to the subject, thereby eliciting an immune response in the subject in need thereof. The antibody treatment may elicit secondary immune responses to the cancer.

Some embodiments provided herein relate to methods of treating cancer and/or a tumor in a subject. In some embodiments, the methods include selecting a treatment subject with cancer and/or a tumor; and administering a disclosed antibody composition to the subject, thereby treating the cancer and/or tumor in the subject.

The foregoing and other features of the disclosure will become more apparent from the following detailed description, which proceeds with reference to the accompanying figures.

### BRIEF DESCRIPTION OF THE DRAWINGS

FIGS. 1A and 1B are schematics illustrating the expression of neoantigens as expressed intracellularly (FIG. 1A) and on the cell surface and their interaction with antibodies specific for the neoantigen. As shown in FIG. 1A, the neoantigens that are expressed intracellularly are not available to interact with antibody until cell lysis. In FIG. 1B, the neoantigens expressed extracellularly on the cell surface and are available for interaction with antibody prior to lysis.

FIG. 2 is a bar graph for the measurement of antibody reactivity to intact or lysed (L) cells.

FIG. 3 is a bar graph of ELISPOT measurements induced by the sera of vaccinated animals in the T-cells (PBMCs) from different animals.

FIGS. 4A-4C are images illustrating staining of B16 Melanoma cells (FIG. 4A), preimmune sera (FIG. 4B), and 4T1 breast cancer cells (FIG. 4C) treated with ∂-6-21.

FIGS. 5A-5C are images illustrating staining of B16 Melanoma cells (FIG. 5A), preimmune sera (FIG. 5B), and 4T1 breast cancer cells (FIG. 5C) treated with ∂-1-78 antibodies.

### DETAILED DESCRIPTION

In the following detailed description, reference is made to the accompanying drawings which form a part hereof, and in which are shown by way of illustration embodiments that may be practiced. It is to be understood that other embodiments may be utilized and structural or logical changes may be made without departing from the scope. Therefore, the following detailed description is not to be taken in a limiting sense, and the scope of embodiments is defined by the appended claims and their equivalents.

Various operations may be described as multiple discrete operations in turn, in a manner that may be helpful in understanding embodiments; however, the order of description should not be construed to imply that these operations are order dependent.

For the purposes of the description, a phrase in the form “A/B” or in the form “A and/or B” means (A), (B), or (A and B). For the purposes of the description, a phrase in the form “at least one of A, B, and C” means (A), (B), (C), (A and B), (A and C), (B and C), or (A, B and C). For the purposes of the description, a phrase in the form “(A)B” means (B) or (AB) that is, A is an optional element.

The description may use the terms “embodiment” or “embodiments,” which may each refer to one or more of the same or different embodiments. Furthermore, the terms “comprising,” “including,” “having,” and the like, as used with respect to embodiments, are synonymous, and are generally intended as “open” terms (e.g., the term “including” should be interpreted as “including but not limited to,” the term “having” should be interpreted as “having at least,” the term “includes” should be interpreted as “includes but is not limited to,” etc.).

With respect to the use of any plural and/or singular terms herein, those having skill in the art can translate from the plural to the singular and/or from the singular to the plural as is appropriate to the context and/or application. The various singular/plural permutations may be expressly set forth herein for sake of clarity.

Unless otherwise noted, technical terms are used according to conventional usage. Definitions of common terms in molecular biology can be found in Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); and Robert A. Meyers (ed.), Molecular Biology and Biotechnology: a Comprehensive Desk Reference, published by VCH Publishers, Inc., 1995 (ISBN 9780471185710); and other similar references. The singular terms “a,” “an,” and “the” include plural referents unless context clearly indicates otherwise. Similarly, the word “or” is intended to include “and” unless the context clearly indicates otherwise. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of this disclosure, suitable methods and materials are described below. All publications, patent applications, patents, and other references mentioned herein are incorporated by reference in their entirety. In case of conflict, the present specification, including explanations of terms, will control. In addition, the materials, methods, and examples are illustrative only and not intended to be limiting.

To facilitate review of the various embodiments of this disclosure, the following explanations of specific terms are provided, along with particular examples:

Antigen: A compound, composition, or substance that can stimulate the production of antibodies or a T cell response in an animal, including compositions that are injected or absorbed into an animal. An antigen reacts with the products of specific humoral or cellular immunity, including those induced by heterologous immunogens, such as the peptides disclosed herein. The term “antigen” includes all related antigenic epitopes. “Epitope” or “antigenic determinant” refers to a site on an antigen to which B and/or T cells respond. Epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5 amino acids in a unique spatial conformation. Methods of determining spatial conformation of epitopes include, for example, x-ray crystallography and 2-dimensional nuclear magnetic resonance.

Antibody: Immunoglobulin molecules and immunologically active portions of immunoglobulin molecules, i.e., molecules that contain an antigen binding site that specifically binds (immunoreacts with) an antigen.

A naturally occurring antibody (e.g., IgG, IgM, IgD) includes four polypeptide chains, two heavy (H) chains and two light (L) chains interconnected by disulfide bonds. However, it has been shown that the antigen-binding function of an antibody can be performed by fragments of a naturally occurring antibody. Thus, these antigen-binding fragments are also intended to be designated by the term “antibody.” Specific, non-limiting examples of binding fragments encompassed within the term antibody include (i) a Fab fragment consisting of the VL, VH, CL and CH1 domains; (ii) an Fd fragment consisting of the VH and CH1 domains; (iii) an Fv fragment consisting of the VL and VH domains of a single arm of an antibody, (iv) a dAb fragment (Ward et al., Nature 341:544-546, 1989) which consists of a VH domain; (v) an isolated complementarity determining region (CDR); and (vi) a F(ab′)2 fragment, a bivalent fragment comprising two Fab fragments linked by a disulfide bridge at the hinge region.

Immunoglobulins and certain variants thereof are known and many have been prepared in recombinant cell culture (e.g., see U.S. Pat. Nos. 4,745,055; 4,444,487; WO 88/03565; EP 256,654; EP 120,694; EP 125,023; Faoulkner et al., Nature 298:286, 1982; Morrison, J. Immunol. 123:793, 1979; Morrison et al., Ann Rev Immunol 2:239, 1984). Humanized antibodies and fully human antibodies are also known in the art.

Animal: Living multi-cellular vertebrate organisms, a category that includes, for example, mammals and birds. The term mammal includes both human and non-human mammals. Similarly, the term “subject” includes both human and veterinary subjects.

Array: An arrangement of molecules, such as biological macromolecules (such as peptides), in addressable locations on or in a substrate. A “microarray” is an array that is miniaturized so as to require or be aided by microscopic examination for evaluation or analysis. The array of molecules (“features”) makes it possible to carry out a very large number of analyses on a sample at one time. Within an array, each arrayed sample is addressable, in that its location can be reliably and consistently determined within at least two dimensions of the array. The feature application location on an array can assume different shapes. For example, the array can be regular (such as arranged in uniform rows and columns) or irregular. Thus, in ordered arrays the location of each sample is assigned to the sample at the time when it is applied to the array, and a key may be provided in order to correlate each location with the appropriate target or feature position. Often, ordered arrays are arranged in a symmetrical grid pattern, but samples can be arranged in other patterns (such as in radially distributed lines, spiral lines, or ordered clusters). Addressable arrays usually are computer readable, in that a computer can be programmed to correlate a particular address on the array with information about the sample at that position (such as hybridization or binding data, including for instance signal intensity). In some examples of computer readable formats, the subject features in the array are arranged regularly, for instance in a Cartesian grid pattern, which can be correlated to address information by a computer.

Binding or stable binding: An association between two substances or molecules, such as the association of an antibody with a peptide. Binding can be detected by any procedure known to one skilled in the art, such as by physical or functional properties of the formed complexes, such as a target/antibody complex.

Diagnostic: Identifying the presence or nature of a pathologic condition. Diagnostic methods differ in their sensitivity and specificity. The “sensitivity” of a diagnostic assay is the percentage of diseased subjects who test positive (percent of true positives). The “specificity” of a diagnostic assay is 1 minus the false positive rate, where the false positive rate is defined as the proportion of those without the disease who test positive. While a particular diagnostic method may not provide a definitive diagnosis of a condition, it suffices if the method provides a positive indication that aids in diagnosis. “Prognostic” means predicting the probability of development (for example, severity) of a pathologic condition.

Frameshift and Frameshift Peptide: A frameshift in the RNA results from an insertion/deletion or fusion/deletion at the DNA or RNA level that results in the shifting of the reading frame of the ribosome. The resultant peptide is out of frame and does not correspond to a normal life sequence peptide. A Frameshift may also result from the inappropriate initiation of translation by the ribosome. The results of these mistakes are Frameshift Peptides (FSPs).

Immune response: A response of a cell of the immune system, such as a B cell, T cell, or monocyte, to a stimulus. In one embodiment, the response is specific for a particular antigen (an “antigen-specific response”).

Immunogenic peptide: A peptide which comprises an allele-specific motif or other sequence such that the peptide will bind an MHC molecule and induce a cytotoxic T lymphocyte (“CTL”) response, or a B cell response (e.g. antibody production) against the antigen from which the immunogenic peptide is derived.

Indel: The insertion or deletion of 1, 2 or multiple of bases that causes the change in reading frame.

“Inhibit,” “inhibiting,” and “inhibition”: To decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

Label: A detectable compound or composition that is conjugated directly or indirectly to another molecule to facilitate detection of that molecule. Specific, non-limiting examples of labels include fluorescent tags, enzymatic linkages, and radioactive isotopes.

Microsatellite: A run in the DNA of the same set of nucleotides. For example, 10 Adenosines in a row (homopolymer) or 10 AGG triplets in a row.

Mis-splicing: If an exon fuses to another exon that is not a natural splicing the resultant protein will be aberrant or a frameshift peptide.

Monoclonal antibody: An antibody that is the product of one B-cell clone. A monoclonal antibody may be produced from hybridomas with B-cells or in bacteria, yeast, or viruses.

Peptide Modifications: Immunogenic peptides include synthetic embodiments of peptides described herein. In addition, analogs (non-peptide organic molecules), derivatives (chemically functionalized peptide molecules obtained starting with the disclosed peptide sequences) and variants (homologs) of these proteins can be utilized in the methods described herein. Each polypeptide of this disclosure is comprised of a sequence of amino acids, which may be either L- and/or D-amino acids, naturally occurring and otherwise.

Peptides can be modified by a variety of chemical techniques to produce derivatives having essentially the same activity as the unmodified peptides, and optionally having other desirable properties. For example, carboxylic acid groups of the protein, whether carboxyl-terminal or side chain, can be provided in the form of a salt of a pharmaceutically-acceptable cation or esterified to form a C1-C16 ester, or converted to an amide of formula NR1R2 wherein R1 and R2 are each independently H or C1-C16 alkyl, or combined to form a heterocyclic ring, such as a 5- or 6-membered ring Amino groups of the peptide, whether amino-terminal or side chain, can be in the form of a pharmaceutically-acceptable acid addition salt, such as the HCl, HBr, acetic, benzoic, toluene sulfonic, maleic, tartaric and other organic salts, or can be modified to C1-C16 alkyl or dialkyl amino or further converted to an amide.

Hydroxyl groups of the peptide side chains may be converted to C1-C16 alkoxy or to a C1-C16 ester using well-recognized techniques. Phenyl and phenolic rings of the peptide side chains may be substituted with one or more halogen atoms, such as fluorine, chlorine, bromine or iodine, or with C1-C16 alkyl, C1-C16 alkoxy, carboxylic acids and esters thereof, or amides of such carboxylic acids. Methylene groups of the peptide side chains can be extended to homologous C2-C4 alkylenes. Thiols can be protected with any one of a number of well-recognized protecting groups, such as acetamide groups. Those skilled in the art will also recognize methods for introducing cyclic structures into the peptides of this disclosure to select and provide conformational constraints to the structure that result in enhanced stability.

Peptidomimetic and organomimetic embodiments are envisioned, whereby the three-dimensional arrangement of the chemical constituents of such peptido- and organomimetics mimic the three-dimensional arrangement of the peptide backbone and component amino acid side chains, resulting in such peptido- and organomimetics of an immunogenic Brachyury polypeptide having measurable or enhanced ability to generate an immune response. For computer modeling applications, a pharmacophore is an idealized three-dimensional definition of the structural requirements for biological activity. Peptido- and organomimetics can be designed to fit each pharmacophore with current computer modeling software (using computer assisted drug design or CADD). See Walters, “Computer-Assisted Modeling of Drugs,” in Klegerman & Groves, eds., 1993, Pharmaceutical Biotechnology, Interpharm Press: Buffalo Grove, Ill., pp. 165-174 and Principles of Pharmacology, Munson (ed.) 1995, Ch. 102, for descriptions of techniques used in CADD. Also included are mimetics prepared using such techniques.

Peptide: Any chain of amino acids, regardless of length or post-translational modification (e.g., glycosylation or phosphorylation). A polypeptide can be between 3 and 30 amino acids in length. In one embodiment, a polypeptide is from about 5 to about 25 amino acids in length. In yet another embodiment, a polypeptide is from about 8 to about 12 amino acids in length. In yet another embodiment, a peptide is about 5 amino acids in length. With regard to polypeptides, the word “about” indicates integer amounts.

Sequence identity: The similarity between amino acid sequences is expressed in terms of the similarity between the sequences, otherwise referred to as sequence identity. Sequence identity is frequently measured in terms of percentage identity (or similarity or homology); the higher the percentage, the more similar the two sequences are. Homologs or variants of a polypeptide will possess a relatively high degree of sequence identity when aligned using standard methods.

Within the context of an immunogenic peptide, a “conserved residue” is one which appears in a significantly higher frequency than would be expected by random distribution at a particular position in a peptide. In one embodiment, a conserved residue is one where the MHC structure may provide a contact point with the immunogenic peptide.

Methods of alignment of sequences for comparison are well known in the art. Various programs and alignment algorithms are described in: Smith and Waterman, Adv. Appl. Math. 2:482, 1981; Needleman and Wunsch, J. Mol. Biol. 48:443, 1970; Higgins and Sharp, Gene 73:237, 1988; Higgins and Sharp, CABIOS 5:151, 1989; Corpet et al., Nucleic Acids Research 16:10881, 1988; and Pearson and Lipman, Proc. Natl. Acad. Sci. USA 85:2444, 1988. Altschul et al., Nature Genet. 6:119, 1994, presents a detailed consideration of sequence alignment methods and homology calculations.

The NCBI Basic Local Alignment Search Tool (BLAST) (Altschul et al., J. Mol. Biol. 215:403, 1990) is available from several sources, including the National Center for Biotechnology Information (NCBI, Bethesda, Md.) and on the internet, for use in connection with the sequence analysis programs blastp, blastn, blastx, tblastn and tblastx. A description of how to determine sequence identity using this program is available on the NCBI website on the internet.

Homologs and variants of a polypeptide are typically characterized by possession of at least 75%, for example at least 80%, sequence identity counted over the full length alignment with the amino acid sequence using the NCBI Blast 2.0, gapped blastp set to default parameters. For comparisons of amino acid sequences of greater than about 30 amino acids, the Blast 2 sequences function is employed using the default BLOSUM62 matrix set to default parameters, (gap existence cost of 11, and a per residue gap cost of 1). When aligning short peptides (fewer than around 30 amino acids), the alignment should be performed using the Blast 2 sequences function, employing the PAM30 matrix set to default parameters (open gap 9, extension gap 1 penalties). Proteins with even greater similarity to the reference sequences will show increasing percentage identities when assessed by this method, such as at least 80%, at least 85%, at least 90%, at least 95%, at least 98%, or at least 99% sequence identity. Methods for determining sequence identity over such short windows are available at the NCBI website on the internet. One of skill in the art will appreciate that these sequence identity ranges are provided for guidance only; it is entirely possible that strongly significant homologs could be obtained that fall outside of the ranges provided.

“Sequences in Life Space”: A phrase referring to a peptide or DNA/RNA sequence which is found in nature in an organism or virus normal functions.

Treatment: A method of reducing the effects of a disease or condition. Treatment can also refer to a method of reducing the disease or condition itself rather than just the symptoms. The treatment can be any reduction from native levels and can be but is not limited to the complete ablation of the disease, condition, or the symptoms of the disease or condition. For example, a disclosed method for reducing the effects of a cancer is considered to be a treatment if there is a 10% reduction in one or more symptoms of the disease (e.g., tumor size) in a subject with the disease when compared to native levels in the same subject or control subjects. Thus, the reduction can be a 10, 20, 30, 40, 50, 60, 70, 80, 90, 100%, or any amount of reduction in between as compared to native or control levels. It is also understood and contemplated herein that treatment can refer to any reduction in the progression of a disease or cancer. Thus, for example, methods of reducing the effects of a cancer is considered to be a treatment if there is a 10% reduction in the tumor growth rate relative to a control subject or tumor growth rates in the same subject prior to the treatment. It is understood that the reduction can be a 10, 20, 30, 40, 50, 60, 70, 80, 90, 100%, or any amount of reduction in between as compared to native or control levels.

Suitable methods and materials for the practice or testing of this disclosure are described below. Such methods and materials are illustrative only and are not intended to be limiting. Other methods and materials similar or equivalent to those described herein can be used. For example, conventional methods well known in the art to which this disclosure pertains are described in various general and more specific references, including, for example, Sambrook et al., Molecular Cloning: A Laboratory Manual, 2d ed., Cold Spring Harbor Laboratory Press, 1989; Sambrook et al., Molecular Cloning: A Laboratory Manual, 3d ed., Cold Spring Harbor Press, 2001; Ausubel et al., Current Protocols in Molecular Biology, Greene Publishing Associates, 1992 (and Supplements to 2000); Ausubel et al., Short Protocols in Molecular Biology: A Compendium of Methods from Current Protocols in Molecular Biology, 4th ed., Wiley & Sons, 1999; Harlow and Lane, Antibodies: A Laboratory Manual, Cold Spring Harbor Laboratory Press, 1990; and Harlow and Lane, Using Antibodies: A Laboratory Manual, Cold Spring Harbor Laboratory Press, 1999. In addition, the materials, methods, and examples are illustrative only and not intended to be limiting.

### Methods of Producing Antibodies

Tumor cells make frameshift peptide (FSP) neoantigens. These arise not only from mutations in the DNA, but much more frequently from mistakes in RNA production and processing. Interestingly, while the FS neoantigens arising from DNA mutations are largely personal, the ones from RNA are recurrent in different patients, i.e. they show consistency across a patient population with a specific tumor type. Unexpectedly, the inventors made the discovery that antibodies to these FSPs can bind the surface of tumor cells producing them. In particular, it is disclosed herein that antibodies to FSPs (and even single amino acid mutation neoantigens) bind specifically to the tumor cells producing them. This is remarkable in itself in that it is surprising that a FSP even if it were presented in an MHC complex on the surface would still be able to bind an antibody to it. One would assume that at least half of the peptide surface would not be accessible. That both FS and even SNV neoantigens are recognized indicates tumor cells are presenting neoantigens in a non-MHC mechanism. Without being bound by theory, evidence indicates that some aberrant cells, including tumor cells, present unfolded proteins on the surface via misfolded MHCII proteins. Since proteins terminating in FSPs can be unfolded, they are prime candidates for presentation on tumor cells.

Based upon these unexpected findings, disclosed herein are methods of producing antibodies, for example, therapeutic antibodies. In some examples, a sample obtained from one or more subjects, such as a blood or serum sample, with a particular condition, such a particular tumor, is screened on an array containing FS peptides, such as all FS peptides potentially produced by tumors. With multiple subjects, the individual samples from each subject can be used to detect a set of FSPs that are reactive to antibodies from each of the subjects. In other words, Antibodies generated in the subjects to the FSPs are detected. FSPs that are reactive in most or even all the patients are chosen, for example to produce one or more potential or actual therapeutic antibodies to one or more of the detected FSPs. A therapeutic antibody or other ligand against the chosen FSP is developed. In certain embodiments, the antibody is a monoclonal antibody. In certain embodiments, multiple antibodies are developed, for example, for use in a multi-antibody cocktail specific for a tumor of interest. In certain embodiments, one or more of the antibodies are tested for the ability to kill tumor cells, for example, with and without attachment of toxins.

In some embodiments, a method of producing an antibody composition for treating a cancer and/or tumor of interest includes identifying a first population of peptides that are immunoreactive with a set of biological samples obtained from a set of test subjects that have been identified as having the cancer and/or tumor of interest. This immunoreactivity could be determined separately and then the results combined to determine a consensus set of peptides for each cancer and/or tumor type tested. Alternatively, the samples could be pooled and tested concurrently for each cancer and/or tumor type. In the disclosed method, a set of biological samples is obtained from multiple individuals to define a population of neo-antigens for each cancer and/or tumor type, for example, biological samples are obtained from at least 2, at least 3, at least 4, at least 5, at least 6, at least 7, at least 8, at least 9, at least 10, at least 15, at least 20, at least 25, at least 50, at least 100 or even more individuals identified as having the cancer and/or tumor type of interest. In some examples, controls, such as samples form subjects who do not have cancer are used to determine which FS peptides are specific to the specific cancer and/or tumor type. Once the neo-antigens for cancer and/or tumor type have been identified, the sequences of these peptide are used to prepare a cancer antibody composition for the cancer and/or tumor type of interest. An important aspect of the disclosed method is that the immunological screens are based on frameshifted (FS) peptides that are produced by tumors and are reactive with the immune system, which is not the case with current protocols which only sequence the RNA to determine if the gene is transcribed. Further, because the disclosed methods use peptides that are identified from multiple test subjects, the inherent bias of a single individual, such as in a personalized therapeutic are diminished. Thus, as opposed to other methods which are tailored to a single subject, the disclosed methods can be used with any individual, much the same way a polio vaccine is. Thus disclosed herein are streamlined, cost effective, and efficacious methods to design and produce anti-cancer antibody based therapeutics for a subject, such as a human or animal subject, such as an antibody specific for a specific cancer and/or tumor type. Antibodies designed using methods herein could also be given to a subject to prevent reoccurrence of a cancer and/or tumor.

However, it is contemplated that antibodies against conventional neoantigens encoded in DNA can also be produced. These would also need to be presented on the surface of the tumor cell. This is less likely than FSP as point mutations do not necessarily disrupt the protein structure to create unfolded proteins, as is the case with FSPs. Also, point mutations generally are personal, with only a few recurrent at low levels in patients. This would make the production of the therapeutic antibodies expensive. Finally, some tumors are mutationally “cold”—i.e., not providing many mutations to choose from. Therefore, it would be much more difficult and less likely successful to create therapeutic antibodies to DNA-encoded neoantigens.

Screening with Array Containing FS peptides

An array containing FS peptides associated with a particular condition can be produced and antibody reactivity is determined using an assay for antibody binding to a peptide. In some embodiments, FS peptides for antibody screening are bound to a substrate, such as a plate, a glass slide, a bead, or other substrate. Assays for antibody binding include but are not limited to ELISA, radioimmunoassay, western blot, surface plasmon resonance, immunostaining, immunoprecipitation, mass spectrometry, phage display, flow cytometry, cytometric bead array, immunohistochemistry, high density array, microarray, and combinations thereof.

To develop antibodies for a specific cancer regardless of the particular subject, a smaller subset of neo-antigens (“frameshift neo-antigens”) can be utilized to develop a cancer therapeutic for an individual cancer and/or tumor type. Because the number of frameshift (FS) neo-antigens is much smaller than the total of all possible neo-antigens it is possible to produce arrays of these FS neo-antigens and screen these FS array against a set of biological samples obtained from subjects having a particular cancer and/or tumor of interest. The inventors have discovered that tumors make indel mutations in most microsatellites and that mis-splicing is also recurrent at the same genes in tumors. The FS peptides are produced by insertion and deletion mutations (indels) occurring in microsatellite regions or by mis-splicing of RNA. There are approximately 14,000 potential FS peptides transcribed from microsatellites in exons and approximately 210,000 potential FS from mis-splicing that meet requirements for length. These genes would be more difficult for a tumor to evolve away from. Therefore, it is only necessary to screen a limited number of approximately 100 to a few thousand FS peptides to determine the immunogenic components for a therapeutic composition for a specific type of cancer and/or tumor.

In some embodiments, the biological sample comprises or is selected from the group consisting of blood, plasma, serum, thymus, bone marrow, spleen, lymph node, bronchoalveolar lavage, breast, central nervous system, cerebrospinal fluid, eye, tears, gastrointestinal tract, saliva, feces, urine, heart, kidney, liver, lung, muscle, pancreas, peripheral nervous system, saliva, skin, thyroid, trachea, and tumor. In some embodiments, the biological sample is blood, serum, plasma, or saliva. In some embodiments, the biological sample comprises cells selected from B cells, T cells, CD4+ T cells, CD8+ T cells, Th17 cells, and combinations thereof. In some embodiments, the biological sample comprises an antibody. In some embodiments, all the biological samples tested are the same type. In other embodiments, the biological samples are different types, such as different types of samples listed above.

In some embodiments, the cancer and/or tumor type comprises Acanthoma, Acinic cell carcinoma, Acoustic neuroma, Acral lentiginous melanoma, Acrospiroma, Acute eosinophilic leukemia, Acute lymphoblastic leukemia, Acute megakaryoblastic leukemia, Acute monocytic leukemia, Acute myeloblastic leukemia with maturation, Acute myeloid dendritic cell leukemia, Acute myeloid leukemia, Acute promyelocytic leukemia, Adamantinoma, Adenocarcinoma, Adenoid cystic carcinoma, Adenoma, Adenomatoid odontogenic tumor, Adrenocortical carcinoma, Adult T-cell leukemia, Aggressive NK-cell leukemia, AIDS-Related Cancers, AIDS-related lymphoma, Alveolar soft part sarcoma, Ameloblastic fibroma, Anal cancer, Anaplastic large cell lymphoma, Anaplastic thyroid cancer, Angioimmunoblastic T-cell lymphoma, Angiomyolipoma, Angiosarcoma, Appendix cancer, Astrocytoma, Atypical teratoid rhabdoid tumor, Basal cell carcinoma, Basal-like carcinoma, B-cell leukemia, B-cell lymphoma, Bellini duct carcinoma, Biliary tract cancer, Bladder cancer, Blastoma, Bone Cancer, Bone tumor, Brain Stem Glioma, Brain Tumor, Breast Cancer, Brenner tumor, Bronchial Tumor, Bronchioloalveolar carcinoma, Brown tumor, Burkitt's lymphoma, Cancer of Unknown Primary Site, Carcinoid Tumor, Carcinoma, Carcinoma in situ, Carcinoma of the penis, Carcinoma of Unknown Primary Site, Carcinosarcoma, Castleman's Disease, Central Nervous System Embryonal Tumor, Cerebellar Astrocytoma, Cerebral Astrocytoma, Cervical Cancer, Cholangiocarcinoma, Chondroma, Chondrosarcoma, Chordoma, Choriocarcinoma, Choroid plexus papilloma, Chronic Lymphocytic Leukemia, Chronic monocytic leukemia, Chronic myelogenous leukemia, Chronic Myeloproliferative Disorder, Chronic neutrophilic leukemia, Clear-cell tumor, Colon Cancer, Colorectal cancer, Craniopharyngioma, Cutaneous T-cell lymphoma, Degos disease, Dermatofibrosarcoma protuberans, Dermoid cyst, Desmoplastic small round cell tumor, Diffuse large B cell lymphoma, Dysembryoplastic neuroepithelial tumor, Embryonal carcinoma, Endodermal sinus tumor, Endometrial cancer, Endometrial Uterine Cancer, Endometrioid tumor, Enteropathy-associated T-cell lymphoma, Ependymoblastoma, Ependymoma, Epithelioid sarcoma, Erythroleukemia, Esophageal cancer, Esthesioneuroblastoma, Ewing Family of Tumor, Ewing Family Sarcoma, Ewing's sarcoma, Extracranial Germ Cell Tumor, Extragonadal Germ Cell Tumor, Extrahepatic Bile Duct Cancer, Extramammary Paget's disease, Fallopian tube cancer, Fetus in fetu, Fibroma, Fibrosarcoma, Follicular lymphoma, Follicular thyroid cancer, Gallbladder Cancer, Gallbladder cancer, Ganglioglioma, Ganglioneuroma, Gastric Cancer, Gastric lymphoma, Gastrointestinal cancer, Gastrointestinal Carcinoid Tumor, Gastrointestinal Stromal Tumor, Gastrointestinal stromal tumor, Germ cell tumor, Germinoma, Gestational choriocarcinoma, Gestational Trophoblastic Tumor, Giant cell tumor of bone, Glioblastoma multiforme, Glioma, Gliomatosis cerebri, Glomus tumor, Glucagonoma, Gonadoblastoma, Granulosa cell tumor, Hairy Cell Leukemia, Hairy cell leukemia, Head and Neck Cancer, Head and neck cancer, Heart cancer, Hemangioblastoma, Hemangiopericytoma, Hemangiosarcoma, Hematological malignancy, Hepatocellular carcinoma, Hepatosplenic T-cell lymphoma, Hereditary breast-ovarian cancer syndrome, Hodgkin Lymphoma, Hodgkin's lymphoma, Hypopharyngeal Cancer, Hypothalamic Glioma, Inflammatory breast cancer, Intraocular Melanoma, Islet cell carcinoma, Islet Cell Tumor, Juvenile myelomonocytic leukemia, Kaposi Sarcoma, Kaposi's sarcoma, Kidney Cancer, Klatskin tumor, Krukenberg tumor, Laryngeal Cancer, Laryngeal cancer, Lentigo maligna melanoma, Leukemia, Lip and Oral Cavity Cancer, Liposarcoma, Lung cancer, Luteoma, Lymphangioma, Lymphangiosarcoma, Lymphoepithelioma, Lymphoid leukemia, Lymphoma, Macroglobulinemia, Malignant Fibrous Histiocytoma, Malignant fibrous histiocytoma, Malignant Fibrous Histiocytoma of Bone, Malignant Glioma, Malignant Mesothelioma, Malignant peripheral nerve sheath tumor, Malignant rhabdoid tumor, Malignant triton tumor, MALT lymphoma, Mantle cell lymphoma, Mast cell leukemia, Mediastinal germ cell tumor, Mediastinal tumor, Medullary thyroid cancer, Medulloblastoma, Medulloepithelioma, Melanoma, Meningioma, Merkel Cell Carcinoma, Mesothelioma, Metastatic Squamous Neck Cancer with Occult Primary, Metastatic urothelial carcinoma, Mixed Mullerian tumor, Monocytic leukemia, Mouth Cancer, Mucinous tumor, Multiple Endocrine Neoplasia Syndrome, Multiple myeloma, Mycosis Fungoides, Myelodysplastic Disease, Myelodysplastic Syndromes, Myeloid leukemia, Myeloid sarcoma, Myeloproliferative Disease, Myxoma, Nasal Cavity Cancer, Nasopharyngeal Cancer, Nasopharyngeal carcinoma, Neoplasm, Neurinoma, Neuroblastoma, Neurofibroma, Neuroma, Nodular melanoma, Non-Hodgkin lymphoma, Nonmelanoma Skin Cancer, Non-Small Cell Lung Cancer, Ocular oncology, Oligoastrocytoma, Oligodendroglioma, Oncocytoma, Optic nerve sheath meningioma, Oral cancer, Oropharyngeal Cancer, Osteosarcoma, Ovarian cancer, Ovarian Epithelial Cancer, Ovarian Germ Cell Tumor, Ovarian Low Malignant Potential Tumor, Paget's disease of the breast, Pancoast tumor, Pancreatic cancer, Papillary thyroid cancer, Papillomatosis, Paraganglioma, Paranasal Sinus Cancer, Parathyroid Cancer, Penile Cancer, Perivascular epithelioid cell tumor, Pharyngeal Cancer, Pheochromocytoma, Pineal Parenchymal Tumor of Intermediate Differentiation, Pineoblastoma, Pituicytoma, Pituitary adenoma, Pituitary tumor, Plasma Cell Neoplasm, Pleuropulmonary blastoma, Polyembryoma, Precursor T-lymphoblastic lymphoma, Primary central nervous system lymphoma, Primary effusion lymphoma, Primary Hepatocellular Cancer, Primary Liver Cancer, Primary peritoneal cancer, Primitive neuroectodermal tumor, Prostate cancer, Pseudomyxoma peritonei, Rectal Cancer, Renal cell carcinoma, Respiratory Tract Carcinoma Involving the NUT Gene on Chromosome 15, Retinoblastoma, Rhabdomyoma, Rhabdomyosarcoma, Richter's transformation, Sacrococcygeal teratoma, Salivary Gland Cancer, Sarcoma, Schwannomatosis, Sebaceous gland carcinoma, Secondary neoplasm, Seminoma, Serous tumor, Sertoli-Leydig cell tumor, Sex cord-stromal tumor, Sezary Syndrome, Signet ring cell carcinoma, Skin Cancer, Small blue round cell tumor, Small cell carcinoma, Small Cell Lung Cancer, Small cell lymphoma, Small intestine cancer, Soft tissue sarcoma, Somatostatinoma, Soot wart, Spinal Cord Tumor, Spinal tumor, Splenic marginal zone lymphoma, Squamous cell carcinoma, Stomach cancer, Superficial spreading melanoma, Supratentorial Primitive Neuroectodermal Tumor, Surface epithelial-stromal tumor, Synovial sarcoma, T-cell acute lymphoblastic leukemia, T-cell large granular lymphocyte leukemia, T-cell leukemia, T-cell lymphoma, T-cell prolymphocytic leukemia, Teratoma, Terminal lymphatic cancer, Testicular cancer, Thecoma, Throat Cancer, Thymic Carcinoma, Thymoma, Thyroid cancer, Transitional Cell Cancer of Renal Pelvis and Ureter, Transitional cell carcinoma, Urachal cancer, Urethral cancer, Urogenital neoplasm, Uterine sarcoma, Uveal melanoma, Vaginal Cancer, Verner Morrison syndrome, Verrucous carcinoma, Visual Pathway Glioma, Vulvar Cancer, Waldenstrom's macroglobulinemia, Warthin's tumor, or Wilms' tumor. Thus, using the methods disclosed herein cancer treatments can be produced for any of the listed cancer and/or tumor types as well as any that are not listed.

In some embodiments, disclosed herein are array platforms that allow for development of peptides suitable for producing antibodies to a specific cancer and/or tumor type. The array platforms comprise a plurality of subject features on the surface of the array, for example in addressable locations. Each feature typically comprises a plurality of subject peptides synthesized in situ on the surface of the array, wherein the molecules are identical within a feature, but the sequence or identity of the molecules differ between features. Such array molecules include the synthesis of large synthetic peptide arrays.

The peptide arrays can include control sequences that match epitopes of well characterized monoclonal antibodies (mAbs). Binding patterns to control sequences and to library peptides can be measured to qualify the arrays and the assay process. Additionally, interwafer signal precision can be determined by testing sample replicates e.g., plasma samples, on arrays from different wafers and calculating the coefficients of variation (CV) for all library peptides. Precision of the measurements of binding signals can be determined as an aggregate of the inter-array, inter-slide, inter-wafer and inter-day variations made on arrays synthesized on wafers of the same batch (within wafer batches). Additionally, precision of measurements can be determined for arrays on wafers of different batches (between wafer batches). In some embodiments, measurements of binding signals can be made within and/or between wafer batches with a precision varying less than 5%, less than 10%, less than 15%, less than 20%, less than 25%, or less than 30%.

The technologies disclosed herein include a photolithographic array synthesis platform that merges semiconductor manufacturing processes and combinatorial chemical synthesis to produce array-based libraries on silicon wafers. By utilizing the tremendous advancements in photolithographic feature patterning, the array synthesis platform is highly-scalable and capable of producing combinatorial peptide libraries with 40 million features on an 8-inch wafer. Photolithographic array synthesis is performed using semiconductor wafer production equipment in a class 10,000 cleanroom to achieve high reproducibility. When the wafer is diced into standard microscope slide dimensions, each slide contains more than 3 million distinct chemical entities.

In some embodiments, arrays with peptide libraries produced by photolithographic technologies disclosed herein are used for immune-based assays. Using a subject's, or multiple subjects, antibody repertoire from a biological sample bound to the arrays, a fluorescence binding profile image of the bound array provides sufficient information to classify which peptides are reactive with an antibody from the subject.

Platforms disclosed herein comprise a selection of frameshift peptides disclosed herein, such as peptides resulting from an insertion or deletion error in transcription of an mRNA or peptides resulting from a splicing error such as a trans-splicing error or a cis-splicing error or a mis-initiation of translation error. In some embodiments, platforms herein comprise frameshift peptides comprise peptides having a sequence selected from all MS FS or MS FS from oncogenes, essential genes, or highly expressed genes. In some embodiments, exemplary frameshift peptides include those provided in U.S. Pat. Nos. 9,732,131 and/or 8,796,414.

In some embodiments, the array is a wafer-based, photolithographic, in situ peptide array produced using reusable masks and automation to obtain arrays of scalable numbers of combinatorial sequence peptides. In some embodiments, the peptide array comprises about 100, about 500, about 1000, about 2000, about 3000, about 4000, about 5,000, about 6000, about 7000, about 8000, about 9000, about 10,000, about 15,000, about 20,000, about 30,000, about 40,000, about 50,000, about 100,000, about 200,000, about 300,000, about 400,000, about 500,000, or more peptides having different sequences. Multiple copies of each of the different sequence peptides can be situated on the wafer at addressable locations known as features.

In some embodiments, the array is a glass slide or nitrocellulose membrane having in vitro synthesized peptides spotted in a predetermined pattern and screened for binding of antibodies in a biological sample, such as obtained from one or more subjects.

In some embodiments, detection of antibody binding on a peptide array poses some challenges that can be addressed by the technologies disclosed herein. Accordingly, in some embodiments, the arrays and methods disclosed herein utilize specific coatings and functional group densities on the surface of the array that can tune the desired properties necessary for performing assays. For example, non-specific antibody binding on a peptide array may be minimized by coating the silicon surface with a moderately hydrophilic monolayer polyethylene glycol (PEG), polyvinyl alcohol, carboxymethyl dextran, and combinations thereof. In some embodiments, the hydrophilic monolayer is homogeneous. Second, synthesized peptides are linked to the silicon surface using a spacer that moves the peptide away from the surface so that the peptide is presented to the antibody in an unhindered orientation.

The variant peptides comprising the collection for screening can be from several sources. They could be peptides known to result from point mutations, frameshifts, deletions/insertions or translocation in tumor DNA. Because these types of mutations are personal and occur infrequently, it would take a large number of peptides to represent all of them. Conventional practice is to determine neo-antigens encoded at the DNA level and then confirm expression at the RNA level. However, the inventors have unexpectedly developed that mutations occur much more frequently at the RNA level only. Since microsatellites in coding regions are predicted and limited in number, one can predict a small set of FS peptides resulting from insertion or deletions during transcription that will produce FS neo-antigens. Therefore, methods herein, in some embodiments, comprise screening frameshift variants formed from 1) insertions or deletions in microsatellites in coding regions or 2) from mis-splicing events either in or between genes that create an out-of-frame fusion. These variants have several attractive features as sources for a cancer vaccine component. First, frameshift variants generally have variant peptide sequences of over more than 8 amino acids.

Peptides are produced and displayed in a number of ways. For example, in some embodiments the peptide candidates are synthesized and spotted on arrays. In some embodiments, arrays have about 100 selected FS peptides. In some embodiments, arrays have about 200 selected FS peptides. In some embodiments, arrays have about 300 selected FS peptides. In some embodiments, arrays have about 400 selected FS peptides. In some embodiments, arrays have about 500 selected FS peptides. In some embodiments, arrays have about 600 selected FS peptides. In some embodiments, arrays have about 700 selected FS peptides. In some embodiments, arrays have about 800 selected FS peptides. In some embodiments, arrays have about 900 selected FS peptides. In some embodiments, arrays have about 1000 selected FS peptides. In some embodiments, arrays have about 10,000 selected FS peptides. In some embodiments, arrays have about 20,000 selected FS peptides. In some embodiments, in-situ synthesis could produce an array having 1,000,000 or more peptides per array, or at least 1000, 10,000 or 100,000 peptides per array.

The methods of producing antibodies as disclosed herein provide much more relevant information for making antibodies and have benefits over the existing methods such as simplicity and cost efficacy. The disclosed methods do not require a biopsy of the tumor with the inherent cost, discomfort and danger. The assay itself would be simpler, less expensive and faster than deep sequencing the tumor DNA. The mutations identified in the DNA are most likely to be the result of point mutations which have a low probability of being produced by a patient, or at least immunogenic. Current sequencing protocols are very poor at identifying insertions/deletions in microsatellites or mis-splicing events. The best source of peptides (FS) would be missed by current sequencing protocols. Sequencing a biopsy will only identify mutations in that part of the tumor. Mutations in other parts will be missed. By methods disclosed herein, all immune reactive parts of the tumor will be identified. The identification of components by sequencing will require the application of proprietary algorithms and other assays to identify potentially produced and immunogenic peptides. In contrast, this information is directly readout from the disclosed assay. While the broadest application may be for cancer, the same procedure would apply for an antibody against other chronic diseases. Antibodies based on the MS FS herein could also eliminate cells that are not tumors but are aberrant for other reasons. Such aberrant, dysfunctional cells play a role in diseases such as diabetes, Alzheimer's disease, aging, autoimmune disease, chronic infections, and other diseases.

Methods herein, in some embodiments, comprise methods of frameshift variant development for inclusion in cancer therapeutic development. Frameshift variants, as referred to herein, are alterations in an mRNA caused by errors in transcription, causing an insertion or deletion (indel) of one or two nucleotides in the mRNA or by mis-splicing of RNA resulting in a change in the amino acids of the resulting protein that are encoded after the frameshift variant. Methods of frameshift variant development herein include but are not limited to mRNA sequencing and array based hybridization. In some embodiments, frameshift peptides are developed by bioinformatics analysis of already available sequence data. FS variants peptides due to indels in MS can be directly inferred from the genome sequence data. The frameshifts of particular value are those created by misprocessing at the RNA level and can include indels created at transcription, mis-splicing of exons or improper initiation of translation by the ribosome.

### Generating and Evaluating Produced Antibodies

The term “antibodies” is used herein in a broad sense and includes both polyclonal and monoclonal antibodies. In addition to intact immunoglobulin molecules, also included in the term “antibodies” are fragments or polymers of those immunoglobulin molecules, and human or humanized versions of immunoglobulin molecules or fragments thereof, as long as they are chosen for their ability to interact with their specific target and bring about the desired outcome, such as inhibition or reduction of tumor growth. The antibodies can be tested for their desired activity using the in vitro assays described herein, or by analogous methods, after which their in vivo therapeutic and/or prophylactic activities are tested according to known clinical testing methods.

As used herein, the term “antibody” encompasses, but is not limited to, whole immunoglobulin (i.e., an intact antibody) of any class. Native antibodies are usually heterotetrameric glycoproteins, composed of two identical light (L) chains and two identical heavy (H) chains. Typically, each light chain is linked to a heavy chain by one covalent disulfide bond, while the number of disulfide linkages varies between the heavy chains of different immunoglobulin isotypes. Each heavy and light chain also has regularly spaced intrachain disulfide bridges. Each heavy chain has at one end a variable domain (V(H)) followed by a number of constant domains. Each light chain has a variable domain at one end (V(L)) and a constant domain at its other end; the constant domain of the light chain is aligned with the first constant domain of the heavy chain, and the light chain variable domain is aligned with the variable domain of the heavy chain. Particular amino acid residues are believed to form an interface between the light and heavy chain variable domains. The light chains of antibodies from any vertebrate species can be assigned to one of two clearly distinct types, called kappa (k) and lambda (l), based on the amino acid sequences of their constant domains. Depending on the amino acid sequence of the constant domain of their heavy chains, immunoglobulins can be assigned to different classes. There are five major classes of human immunoglobulins: IgA, IgD, IgE, IgG and IgM, and several of these may be further divided into subclasses (isotypes), e.g., IgG-I, IgG-2, IgG-3, and IgG-4; IgA-I and IgA-2. One skilled in the art would recognize the comparable classes for mouse. The heavy chain constant domains that correspond to the different classes of immunoglobulins are called alpha, delta, epsilon, gamma, and mu, respectively.

The term “variable” is used herein to describe certain portions of the variable domains that differ in sequence among antibodies and are used in the binding and specificity of each particular antibody for its particular antigen. However, the variability is not usually evenly distributed through the variable domains of antibodies. It is typically concentrated in three segments called complementarity determining regions (CDRs) or hypervariable regions both in the light chain and the heavy chain variable domains. The more highly conserved portions of the variable domains are called the framework (FR). The variable domains of native heavy and light chains each comprise four FR regions, largely adopting a b-sheet configuration, connected by three CDRs, which form loops connecting, and in some cases forming part of, the b-sheet structure. The CDRs in each chain are held together in close proximity by the FR regions and, with the CDRs from the other chain, contribute to the formation of the antigen binding site of antibodies (see Kabat E. A. et al., “Sequences of Proteins of Immunological Interest,” National Institutes of Health, Bethesda, Md.). The constant domains are not involved directly in binding an antibody to an antigen, but exhibit various effector functions, such as participation of the antibody in antibody-dependent cellular toxicity.

As used herein, the term “antibody or fragments thereof encompasses chimeric antibodies and hybrid antibodies, with dual or multiple antigen or epitope specificities, and fragments, such as F(ab′)2, Fab′, Fab, sFv, scFv, and the like, including hybrid fragments. Thus, fragments of the antibodies that retain the ability to bind their specific antigens are provided. For example, fragments of antibodies which maintain desired binding activity are included within the meaning of the term “antibody or fragment thereof.” Such antibodies and fragments can be made by techniques known in the art and can be screened for specificity and activity according to the methods set forth in the Examples and in general methods for producing antibodies and screening antibodies for specificity and activity (See Harlow and Lane. Antibodies, A Laboratory Manual. Cold Spring Harbor Publications, New York, (1988)).

Also included within the meaning of “antibody or fragments thereof are conjugates of antibody fragments and antigen binding proteins (single chain antibodies) as described, for example, in U.S. Pat. No. 4,704,692, the contents of which are hereby incorporated by reference.

The fragments, whether attached to other sequences or not, can also include insertions, deletions, substitutions, or other selected modifications of particular regions or specific amino acids residues, provided the activity of the antibody or antibody fragment is not significantly altered or impaired compared to the non-modified antibody or antibody fragment. These modifications can provide for some additional property, such as to remove/add amino acids capable of disulfide bonding, to increase its bio-longevity, to alter its secretory characteristics, etc. In any case, the antibody or antibody fragment must possess a bioactive property, such as specific binding to its cognate antigen. Functional or active regions of the antibody or antibody fragment may be identified by mutagenesis of a specific region of the protein, followed by expression and testing of the expressed polypeptide. Such methods are readily apparent to a skilled practitioner in the art and can include site-specific mutagenesis of the nucleic acid encoding the antibody or antibody fragment. (Zoller, M J. Curr. Opin. Biotechnol. 3:348-354, 1992).

The term “monoclonal antibody” as used herein refers to an antibody obtained from a substantially homogeneous population of antibodies, i.e., the individual antibodies within the population are identical except for possible naturally occurring mutations that may be present in a small subset of the antibody molecules. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, as long as they exhibit the desired antagonistic activity (See, U.S. Pat. No. 4,816,567 and Morrison et al., Proc. Natl. Acad. Sd. USA, 81:6851-6855 (1984)).

The disclosed monoclonal antibodies can be made using any procedure which produces monoclonal antibodies. For example, disclosed monoclonal antibodies can be prepared using hybridoma methods, such as those described by Kohler and Milstein, Nature, 256:495 (1975). In a hybridoma method, a mouse or other appropriate host animal is typically immunized with an immunizing agent to elicit lymphocytes that produce or are capable of producing antibodies that will specifically bind to the immunizing agent. Alternatively, the lymphocytes may be immunized in vitro, e.g., using the HIV Env-CD4-co-receptor complexes described herein.

The monoclonal antibodies may also be made by recombinant DNA methods, such as those described in U.S. Pat. No. 4,816,567 (Cabilly et al.). DNA encoding the disclosed monoclonal antibodies can be readily isolated and sequenced using conventional procedures (e.g., by using oligonucleotide probes that are capable of binding specifically to genes encoding the heavy and light chains of murine antibodies). Libraries of antibodies or active antibody fragments can also be generated and screened using phage display techniques, e.g., as described in U.S. Pat. No. 5,804,440 to Burton et al. and U.S. Pat. No. 6,096,441 to Barbas et al.

In vitro methods are also suitable for preparing monovalent antibodies. Digestion of antibodies to produce fragments thereof, particularly, Fab fragments, can be accomplished using routine techniques known in the art. For instance, digestion can be performed using papain. Examples of papain digestion are described in WO 94/29348 published Dec. 22, 1994 and U.S. Pat. No. 4,342,566. Papain digestion of antibodies typically produces two identical antigen binding fragments, called Fab fragments, each with a single antigen binding site, and a residual Fc fragment. Pepsin treatment yields a fragment that has two antigen combining sites and is still capable of cross-linking antigen.

As used herein, the term “antibody” or “antibodies” can also refer to a human antibody and/or a humanized antibody. Many non-human antibodies (e.g., those derived from mice, rats, or rabbits) are naturally antigenic in humans, and thus can give rise to undesirable immune responses when administered to humans Therefore, the use of human or humanized antibodies in the methods serves to lessen the chance that an antibody administered to a human will evoke an undesirable immune response.

The disclosed human antibodies can be prepared using any technique. Examples of techniques for human monoclonal antibody production include those described by Cole et al. (Monoclonal Antibodies and Cancer Therapy, Alan R. Liss, p. 77, 1985) and by Boemer et al. (J. Immunol., 147(1):86-95, 1991). Human antibodies (and fragments thereof) can also be produced using phage display libraries (Hoogenboom et al., J. Mol. Biol, 227:381, 1991; Marks et al., J Mol. Biol, 222:581, 1991).

The disclosed human antibodies can also be obtained from transgenic animals For example, transgenic, mutant mice that are capable of producing a full repertoire of human antibodies, in response to immunization, have been described (see, e.g., Jakobovits et al., Proc. Natl Acad. ScL USA, 90:2551-255 (1993); Jakobovits et al., Nature, 362:255-258 (1993); Bruggermann et al., Year in Immunol., 7:33 (1993)). Specifically, the homozygous deletion of the antibody heavy chain joining region (J(H)) gene in these chimeric and germ-line mutant mice results in complete inhibition of endogenous antibody production, and the successful transfer of the human germ-line antibody gene array into such germ-line mutant mice results in the production of human antibodies upon antigen challenge. Antibodies having the desired activity are selected using Env-CD4-co-receptor complexes as described herein.

Antibody humanization techniques generally involve the use of recombinant DNA technology to manipulate the DNA sequence encoding one or more polypeptide chains of an antibody molecule. Accordingly, a humanized form of a non-human antibody (or a fragment thereof) is a chimeric antibody or antibody chain (or a fragment thereof, such as an Fv, Fab, Fab′, or other antigen-binding portion of an antibody) which contains a portion of an antigen binding site from a non-human (donor) antibody integrated into the framework of a human (recipient) antibody.

To generate a humanized antibody, residues from one or more complementarity determining regions (CDRs) of a recipient (human) antibody molecule are replaced by residues from one or more CDRs of a donor (non-human) antibody molecule that is known to have desired antigen binding characteristics (e.g., a certain level of specificity and affinity for the target antigen), in some instances, Fv framework (FR) residues of the human antibody are replaced by corresponding non-human residues. Humanized antibodies may also contain residues which are found neither in the recipient antibody nor in the imported CDR or framework sequences. Generally, a humanized antibody has one or more amino acid residues introduced into it from a source which is non-human, in practice, humanized antibodies are typically human antibodies in which some CDR residues and possibly some FR residues are substituted by residues from analogous sites in rodent antibodies. Humanized antibodies generally contain at least a portion of an antibody constant region (Fc), typically that of a human antibody (Jones et al., Nature, 321:522-525 (1986), Reichmann et al., Nature, 332:323-327 (1988), and Presta, Curr. Opin. Struct. Biol, 2:593-596 (1992)).

Methods for humanizing non-human antibodies are well known in the art. For example, humanized antibodies can be generated according to the methods of Winter and co-workers (Jones et al., Nature, 321:522-525 (1986), Riechmann et al., Nature, 332:323-327 (1988), Verhoeyen et al., Science, 239:1534-1536 (1988)), by substituting rodent CDRs or CDR sequences for the corresponding sequences of a human antibody. Methods that can be used to produce humanized antibodies are also described in U.S. Pat. No. 4,816,567 (Cabilly et al.), U.S. Pat. No. 5,565,332 (Hoogenboom et al.), U.S. Pat. No. 5,721,367 (Kay et al.), U.S. Pat. No. 5,837,243 (Deo et al.), U.S. Pat. No. 5,939,598 (Kucherlapati et al.), U.S. Pat. No. 6,130,364 (Jakobovits et al.), and U.S. Pat. No. 6,180,377 (Morgan et al.).

Administration of the antibodies can be done as disclosed herein. Nucleic acid approaches for antibody delivery also exist. The broadly neutralizing antibodies and antibody fragments can also be administered to patients or subjects as a nucleic acid preparation (e.g., DNA or RNA) that encodes the antibody or antibody fragment, such that the patient's or subject's own cells take up the nucleic acid and produce and secrete the encoded antibody or antibody fragment. The delivery of the nucleic acid can be by any means, as disclosed herein, for example.

It is understood that the antibodies disclosed herein can be combined with other agents, molecules, or compounds to increase binding, elicit additional immune responses, or deliver toxic effects to the proximity of the target antigen, e.g., to cells that express the frameshift mutation. Such combinations can occur through the formation of fusion constructs, immunoconjugates, or other combination platform known in the art. Thus, it is understood that the antibodies disclosed herein can be combined with a toxin such as diphtheria toxin, ricin toxin, tetanus toxoid, botulinum toxin, or any other toxin as a fusion construct to form an antibody-toxin fusion. For example, the antibody-toxin fusion construct can comprise a disclosed antibody fused to a diphtheria toxin. It is understood herein that the disclosed toxins such as tetanus and diphtheria can comprise truncation mutants to avoid the antibody response from previous exposure to the toxin. For example, a diphtheria toxin can comprise a truncation mutant diphtheria toxin wherein the toxin comprises a 145-152 amino acid truncation of the c-terminal end of the diphtheria toxin.

### Antibody Compositions and Formulations

Disclosed herein are antibody compositions. Antibody compositions disclosed herein generally comprise a biologically acceptable formulation, gaseous, liquid or solid, or mixture thereof, which are suitable for one or more routes of administration, in vivo delivery or contact. Such formulations include solvents (aqueous or non-aqueous), solutions (aqueous or non-aqueous), emulsions (e.g., oil-in-water or water-in-oil), suspensions, syrups, elixirs, dispersion and suspension media, coatings, isotonic and absorption promoting or delaying agents, compatible with pharmaceutical administration or in vivo contact or delivery. Aqueous and non-aqueous solvents, solutions and suspensions may include suspending agents and thickening agents. Such pharmaceutically acceptable carriers include tablets (coated or uncoated), capsules (hard or soft), microbeads, powder, granules and crystals. Supplementary active compounds (e.g., preservatives, antibacterial, antiviral and antifungal agents) can also be incorporated into the compositions.

To increase an immune response and efficacy in treating cancer, the compositions are optionally coupled to another protein such as ovalbumin or keyhole limpet hemocyanin (KLH), thyroglobulin or a toxin such as tetanus or cholera toxin. In some embodiments the compositions herein can be given with, such as separately or mixed with adjuvants and/or checkpoint inhibitors. Adjuvants include, for example: ABM2, AS01B, AS02, AS02A, Adjumer, Adjuvax, Algammulin, Alum, Aluminum phosphate, Aluminum potassium sulfate, Bordetella pertussis, Calcitriol, Chitosan, Cholera toxin, CpG, Dibutyl phthalate, Dimethyldioctadecylammonium bromide (DDA), Freund's adjuvant, Freund's complete, Freund's incomplete (IFA), GM-CSF, GMDP, Gamma Inulin, Glycerol, HBSS (Hank's Balanced Salt Solution), IL-12, IL-2, Imiquimod, Interferon-Gamma, ISCOM, Lipid Core Peptide (LCP), Lipofectin, Lipopolysaccharide (LPS), Liposomes, MF59, MLP+TDM, Monophosphoryl lipid A, Montanide IMS-1313, Montanide ISA 206, Montanide ISA 720, Montanide ISA-51, Montanide ISA-50, nor-MDP, Oil-in-water emulsion, P1005 (non-ionic copolymer), Pam3Cys (lipoprotein), Pertussis toxin, Poloxamer, QS21, RaLPS, Ribi, Saponin, Seppic ISA 720, Soybean Oil, Squalene, Syntex Adjuvant Formulation (SAF), Synthetic polynucleotides (poly IC/poly AU), TiterMax Tomatine, Vaxfectin, XtendIII, and Zymosan.

Checkpoint inhibitors are inhibitors of proteins known to potentiate or inhibit an immune response and accordingly a checkpoint inhibitor increases the immune response. In some instances, tumors are known to upregulate checkpoint inhibitors to evade the immune response in a patient and checkpoint inhibitors allow the immune system of the patient to recognize and eliminate the tumor. Checkpoint inhibitors herein include but are not limited to a PD-1 inhibitor, a PD-L1 inhibitor, a CTLA-4 inhibitor, Pembrolizumab, Nivolumab, or Atezolizumab. They may be administered systemically or locally as an adjuvant.

Cosolvents may be added to a composition or formulation. Non-limiting examples of cosolvents contain hydroxyl groups or other polar groups, for example, alcohols, such as isopropyl alcohol; glycols, such as propylene glycol, polyethyleneglycol, polypropylene glycol, glycol ether; glycerol; polyoxyethylene alcohols and polyoxyethylene fatty acid esters. Non-limiting examples of cosolvents contain hydroxyl groups or other polar groups, for example, alcohols, such as isopropyl alcohol; glycols, such as propylene glycol, polyethyleneglycol, polypropylene glycol, glycol ether; glycerol; polyoxyethylene alcohols and polyoxyethylene fatty acid esters.

Supplementary compounds (e.g., preservatives, antioxidants, antimicrobial agents including biocides and biostats such as antibacterial, antiviral and antifungal agents) can also be incorporated into the compositions. Pharmaceutical compositions may therefore include preservatives, anti-oxidants and antimicrobial agents.

Preservatives can be used to inhibit microbial growth or increase stability of ingredients thereby prolonging the shelf life of the pharmaceutical formulation. Suitable preservatives are known in the art and include, for example, EDTA, EGTA, benzalkonium chloride or benzoic acid or benzoates, such as sodium benzoate. Antioxidants include, for example, ascorbic acid, vitamin A, vitamin E, tocopherols, and similar vitamins or provitamins

Pharmaceutical compositions can be formulated to be compatible with a particular route of administration. Thus, pharmaceutical compositions include carriers, diluents, or excipients suitable for administration by various routes.

The compositions, including antibodies, can be used therapeutically in combination with a pharmaceutically acceptable carrier. Suitable carriers and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders maybe desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

### Methods of Treatment

Provided herein is a method of eliciting an immune response in a subject in need thereof. In embodiments, the method includes selecting a subject with cancer or at risk for developing cancer. In embodiments, the method includes administering a disclosed composition to the subject, thereby eliciting an immune response in the subject in need thereof.

Disclosed is a method of treating cancer and/or a tumor in a subject. In embodiments, the method includes selecting a treatment subject with cancer and/or a tumor. In embodiments, the method includes administering s disclosed composition to the subject, thereby treating the cancer and/or tumor in the subject.

Methods herein comprise administration of one or more antibodies. In some embodiments, the methods are used to treat a cancer and/or tumor type selected from Acanthoma, Acinic cell carcinoma, Acoustic neuroma, Acral lentiginous melanoma, Acrospiroma, Acute eosinophilic leukemia, Acute lymphoblastic leukemia, Acute megakaryoblastic leukemia, Acute monocytic leukemia, Acute myeloblastic leukemia with maturation, Acute myeloid dendritic cell leukemia, Acute myeloid leukemia, Acute promyelocytic leukemia, Adamantinoma, Adenocarcinoma, Adenoid cystic carcinoma, Adenoma, Adenomatoid odontogenic tumor, Adrenocortical carcinoma, Adult T-cell leukemia, Aggressive NK-cell leukemia, AIDS-Related Cancers, AIDS-related lymphoma, Alveolar soft part sarcoma, Ameloblastic fibroma, Anal cancer, Anaplastic large cell lymphoma, Anaplastic thyroid cancer, Angioimmunoblastic T-cell lymphoma, Angiomyolipoma, Angiosarcoma, Appendix cancer, Astrocytoma, Atypical teratoid rhabdoid tumor, Basal cell carcinoma, Basal-like carcinoma, B-cell leukemia, B-cell lymphoma, Bellini duct carcinoma, Biliary tract cancer, Bladder cancer, Blastoma, Bone Cancer, Bone tumor, Brain Stem Glioma, Brain Tumor, Breast Cancer, Brenner tumor, Bronchial Tumor, Bronchioloalveolar carcinoma, Brown tumor, Burkitt' s lymphoma, Cancer of Unknown Primary Site, Carcinoid Tumor, Carcinoma, Carcinoma in situ, Carcinoma of the penis, Carcinoma of Unknown Primary Site, Carcinosarcoma, Castleman's Disease, Central Nervous System Embryonal Tumor, Cerebellar Astrocytoma, Cerebral Astrocytoma, Cervical Cancer, Cholangiocarcinoma, Chondroma, Chondrosarcoma, Chordoma, Choriocarcinoma, Choroid plexus papilloma, Chronic Lymphocytic Leukemia, Chronic monocytic leukemia, Chronic myelogenous leukemia, Chronic Myeloproliferative Disorder, Chronic neutrophilic leukemia, Clear-cell tumor, Colon Cancer, Colorectal cancer, Craniopharyngioma, Cutaneous T-cell lymphoma, Degos disease, Dermatofibrosarcoma protuberans, Dermoid cyst, Desmoplastic small round cell tumor, Diffuse large B cell lymphoma, Dysembryoplastic neuroepithelial tumor, Embryonal carcinoma, Endodermal sinus tumor, Endometrial cancer, Endometrial Uterine Cancer, Endometrioid tumor, Enteropathy-associated T-cell lymphoma, Ependymoblastoma, Ependymoma, Epithelioid sarcoma, Erythroleukemia, Esophageal cancer, Esthesioneuroblastoma, Ewing Family of Tumor, Ewing Family Sarcoma, Ewing's sarcoma, Extracranial Germ Cell Tumor, Extragonadal Germ Cell Tumor, Extrahepatic Bile Duct Cancer, Extramammary Paget's disease, Fallopian tube cancer, Fetus in fetu, Fibroma, Fibrosarcoma, Follicular lymphoma, Follicular thyroid cancer, Gallbladder Cancer, Gallbladder cancer, Ganglioglioma, Ganglioneuroma, Gastric Cancer, Gastric lymphoma, Gastrointestinal cancer, Gastrointestinal Carcinoid Tumor, Gastrointestinal Stromal Tumor, Gastrointestinal stromal tumor, Germ cell tumor, Germinoma, Gestational choriocarcinoma, Gestational Trophoblastic Tumor, Giant cell tumor of bone, Glioblastoma multiforme, Glioma, Gliomatosis cerebri, Glomus tumor, Glucagonoma, Gonadoblastoma, Granulosa cell tumor, Hairy Cell Leukemia, Hairy cell leukemia, Head and Neck Cancer, Head and neck cancer, Heart cancer, Hemangioblastoma, Hemangiopericytoma, Hemangiosarcoma, Hematological malignancy, Hepatocellular carcinoma, Hepatosplenic T-cell lymphoma, Hereditary breast-ovarian cancer syndrome, Hodgkin Lymphoma, Hodgkin's lymphoma, Hypopharyngeal Cancer, Hypothalamic Glioma, Inflammatory breast cancer, Intraocular Melanoma, Islet cell carcinoma, Islet Cell Tumor, Juvenile myelomonocytic leukemia, Kaposi Sarcoma, Kaposi's sarcoma, Kidney Cancer, Klatskin tumor, Krukenberg tumor, Laryngeal Cancer, Laryngeal cancer, Lentigo maligna melanoma, Leukemia, Lip and Oral Cavity Cancer, Liposarcoma, Lung cancer, Luteoma, Lymphangioma, Lymphangiosarcoma, Lymphoepithelioma, Lymphoid leukemia, Lymphoma, Macroglobulinemia, Malignant Fibrous Histiocytoma, Malignant fibrous histiocytoma, Malignant Fibrous Histiocytoma of Bone, Malignant Glioma, Malignant Mesothelioma, Malignant peripheral nerve sheath tumor, Malignant rhabdoid tumor, Malignant triton tumor, MALT lymphoma, Mantle cell lymphoma, Mast cell leukemia, Mediastinal germ cell tumor, Mediastinal tumor, Medullary thyroid cancer, Medulloblastoma, Medulloepithelioma, Melanoma, Meningioma, Merkel Cell Carcinoma, Mesothelioma, Metastatic Squamous Neck Cancer with Occult Primary, Metastatic urothelial carcinoma, Mixed Mullerian tumor, Monocytic leukemia, Mouth Cancer, Mucinous tumor, Multiple Endocrine Neoplasia Syndrome, Multiple myeloma, Mycosis Fungoides, Myelodysplastic Disease, Myelodysplastic Syndromes, Myeloid leukemia, Myeloid sarcoma, Myeloproliferative Disease, Myxoma, Nasal Cavity Cancer, Nasopharyngeal Cancer, Nasopharyngeal carcinoma, Neoplasm, Neurinoma, Neuroblastoma, Neurofibroma, Neuroma, Nodular melanoma, Non-Hodgkin lymphoma, Nonmelanoma Skin Cancer, Non-Small Cell Lung Cancer, Ocular oncology, Oligoastrocytoma, Oligodendroglioma, Oncocytoma, Optic nerve sheath meningioma, Oral cancer, Oropharyngeal Cancer, Osteosarcoma, Ovarian cancer, Ovarian Epithelial Cancer, Ovarian Germ Cell Tumor, Ovarian Low Malignant Potential Tumor, Paget's disease of the breast, Pancoast tumor, Pancreatic cancer, Papillary thyroid cancer, Papillomatosis, Paraganglioma, Paranasal Sinus Cancer, Parathyroid Cancer, Penile Cancer, Perivascular epithelioid cell tumor, Pharyngeal Cancer, Pheochromocytoma, Pineal Parenchymal Tumor of Intermediate Differentiation, Pineoblastoma, Pituicytoma, Pituitary adenoma, Pituitary tumor, Plasma Cell Neoplasm, Pleuropulmonary blastoma, Polyembryoma, Precursor T-lymphoblastic lymphoma, Primary central nervous system lymphoma, Primary effusion lymphoma, Primary Hepatocellular Cancer, Primary Liver Cancer, Primary peritoneal cancer, Primitive neuroectodermal tumor, Prostate cancer, Pseudomyxoma peritonei, Rectal Cancer, Renal cell carcinoma, Respiratory Tract Carcinoma Involving the NUT Gene on Chromosome 15, Retinoblastoma, Rhabdomyoma, Rhabdomyosarcoma, Richter's transformation, Sacrococcygeal teratoma, Salivary Gland Cancer, Sarcoma, Schwannomatosis, Sebaceous gland carcinoma, Secondary neoplasm, Seminoma, Serous tumor, Sertoli-Leydig cell tumor, Sex cord-stromal tumor, Sezary Syndrome, Signet ring cell carcinoma, Skin Cancer, Small blue round cell tumor, Small cell carcinoma, Small Cell Lung Cancer, Small cell lymphoma, Small intestine cancer, Soft tissue sarcoma, Somatostatinoma, Soot wart, Spinal Cord Tumor, Spinal tumor, Splenic marginal zone lymphoma, Squamous cell carcinoma, Stomach cancer, Superficial spreading melanoma, Supratentorial Primitive Neuroectodermal Tumor, Surface epithelial-stromal tumor, Synovial sarcoma, T-cell acute lymphoblastic leukemia, T-cell large granular lymphocyte leukemia, T-cell leukemia, T-cell lymphoma, T-cell prolymphocytic leukemia, Teratoma, Terminal lymphatic cancer, Testicular cancer, Thecoma, Throat Cancer, Thymic Carcinoma, Thymoma, Thyroid cancer, Transitional Cell Cancer of Renal Pelvis and Ureter, Transitional cell carcinoma, Urachal cancer, Urethral cancer, Urogenital neoplasm, Uterine sarcoma, Uveal melanoma, Vaginal Cancer, Verner Morrison syndrome, Verrucous carcinoma, Visual Pathway Glioma, Vulvar Cancer, Waldenstrom's macroglobulinemia, Warthin's tumor, or Wilms' tumor.

Effective dosages and schedules for administering the compositions may be determined empirically, and making such determinations is within the skill in the art. The dosage ranges for the administration of the compositions are those large enough to produce the desired effect in which the symptoms/disorder are/is affected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counter indications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone might range from about 1 mg/kg to up to 100 mg/kg of body weight or more per day, depending on the factors mentioned above.

Following administration of a disclosed composition, such as an antibody, for treating, inhibiting, or preventing a cancer, the efficacy of the y or prophylaxis can be assessed in various ways well known to the skilled practitioner. For instance, one of ordinary skill in the art will understand that a composition, such as an antibody, disclosed herein is efficacious in treating, inhibiting, or preventing a cancer in a subject by observing that the composition reduces tumor growth or prevents a further increase in tumor size.

### Kits

The present disclosure also provides kits for treating a specific condition, such as a specific type of cancer. Such kits may include one or more containers comprising a formulation as described herein, which comprises a disclosed antibody composition.

In some embodiments, the kit may comprise instructions for use in accordance with any of the methods described herein. The included instructions may comprise a description of administration of the formulation according to any of the methods described herein. The kit may further comprise a description of selecting an individual suitable for treatment based on identifying whether that individual has or is at risk of acquiring a particular condition, including cancer.

The instructions relating to the use generally include information as to dosage, dosing schedule, and route of administration for the intended treatment. The containers may be unit doses, bulk packages (e.g., multi-dose packages) or sub-unit doses. Instructions supplied in the kits of the invention are typically written instructions on a label or package insert (e.g., a paper sheet included in the kit), but machine-readable instructions (e.g., instructions carried on a magnetic or optical storage disk) are also acceptable. The label or package insert indicates that the composition is used for a particular condition or disease, including treating cancer. Instructions may be provided for practicing any of the methods described herein.

The kits of this invention are in suitable packaging. Suitable packaging includes, but is not limited to, vials, bottles, jars, flexible packaging (e.g., sealed Mylar or plastic bags), and the like. Kits may optionally provide additional components such as interpretive information. Normally, the kit comprises a container and a label or package insert(s) on or associated with the container. In some embodiments, the invention provides articles of manufacture comprising contents of the kits described above.

The following examples are provided to illustrate particular features of certain embodiments. However, the particular features described below should not be construed as limitations on the scope of the disclosure, but rather as examples from which equivalents will be recognized by those of ordinary skill in the art.

### EXAMPLE

Patients with a particular type of cancer, e.g., breast cancer, will be screened for having antibodies to a frameshift peptides. Frameshift peptides that patients with breast cancer often react to will be chosen. Monoclonal antibodies to a chosen set of these peptides will be selected. The criteria for choice can include frequency of reactivity in the patients or the level of antibody reactivity, for example. The monoclonal antibodies can be tested in vitro for their ability to bind and or kill cancer cells (cell lines or tumor cells) that express the frameshifted RNAs. These candidates can be further screened in mouse tumor models for therapeutic effects when injected. Based on this data, one or more of the antibodies can be produced on large scale by Good Manufacturing Practice to enter into clinical trials in patients with the relevant cancer. If the antibody is shown to be safe and efficacious it could be widely used to treat cancer.

The following data is provided to support the feasibility of the disclosed method. The data demonstrates in multiple ways that frameshift peptides (FSP) are on the surface of tumor cells, are accessible to antibodies against them and cause tumor cells to be killed.

### Antibody Mediating Tumor Killing

Tumor line ID8 was used for the following studies. ELISA plates were coated with 1×105 tumor cells/well (PBS-Carbonate buffer); release cells (whole cell+lysate cells): and treated (cell lysate). Antibodies were present at a 1/200 dilution; tumor vaccinated mice (Tvac) and tumor at end point (Tep).

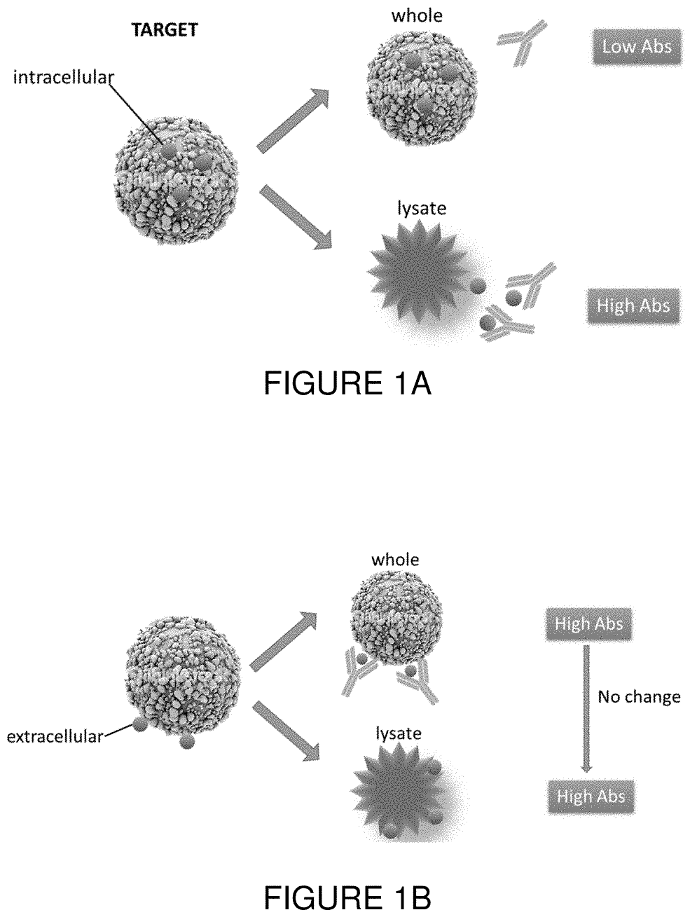

FIGS. 1A and 1B are schematics illustrating the basis for the experiments to determine if the FSP are on the surface of tumor cells and accessible to antibodies. If the FSP are only expressed internal to the cell there will be more antibody binding in lysed cells than in intact cells (FIG. 1A). If the FSPs are expressed in the outside of the cell the amount of antibody binding will be approximately equal for intact and lysed cells (FIG. 1B). The sera was created by vaccination with 8 FSPs or 10 DNA neoepitopes.

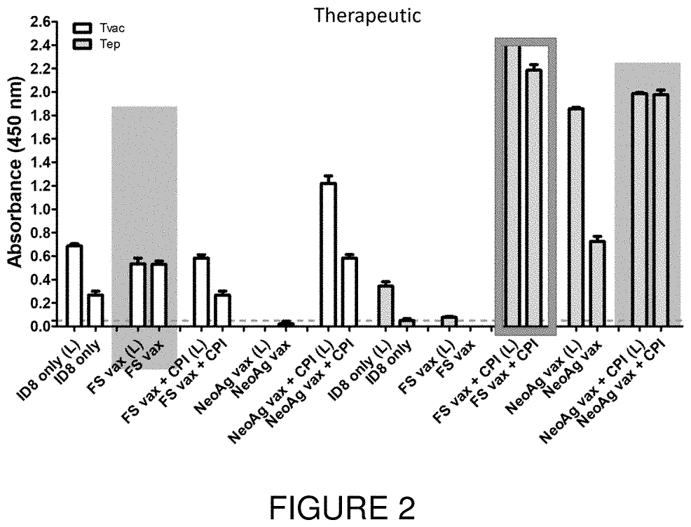

FIG. 2 is a bar graph for the measurement of antibody reactivity to intact or lysed (L) cells. ID8 tumor cells were treated with sera from mice with tumors at the end of vaccination (Tvac) or when they were sacrificed at the end of the experiment. The absorbance is proportional to antibody binding. The important measure is the relative binding of intact and lysed cells. As highlighted with boxes, the FS vaccinated mice, at both timepoints, had equal antibody reactivity for intact and lysed cells. This indicates that the FSP are mostly on the surface of the tumor cells. Mice were also vaccinated with 10 DNA mutation neoepitopes that occur in this cell line. These epitopes were on the surface at the end of the experiment, but not earlier.

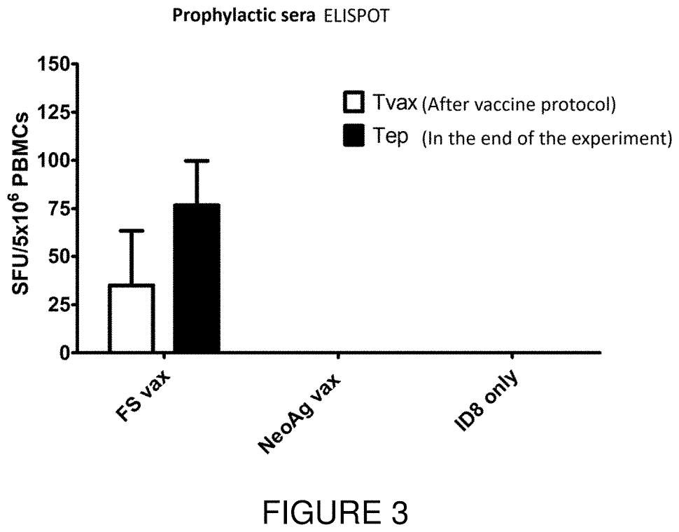

FIG. 3 is a bar graph of ELISPOT measurements induced by the sera of vaccinated animals in the T-cells (PBMCs) from different animals. It can be seen that only animals vaccinated with the FSPs produced T-cells stimulated by the antibodies. This indicates that the antibodies can stimulate T-cell killing of the tumor cells.

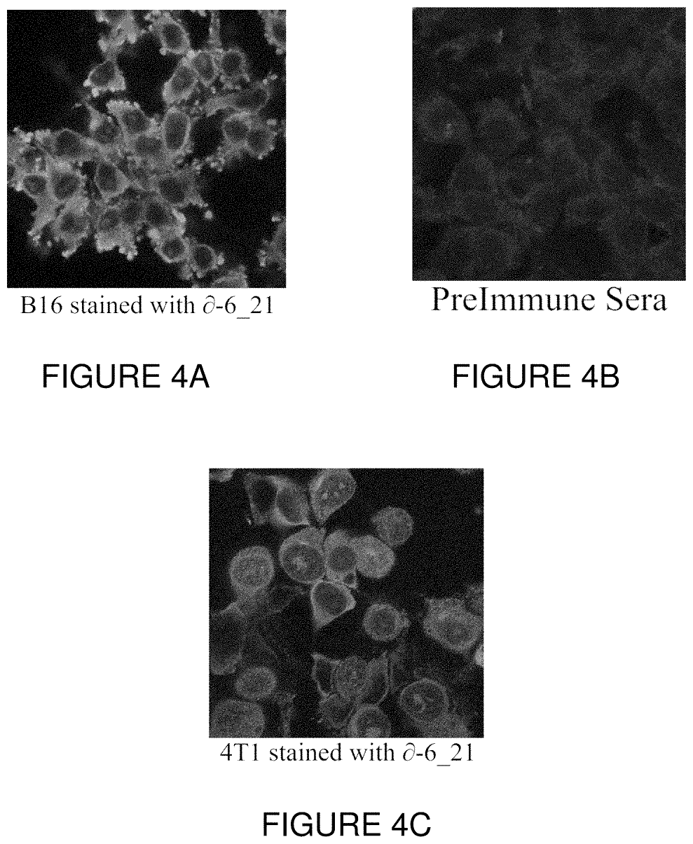

FIGS. 4A-4C demonstrate that the FSP are on the outside of tumor cells. Antisera was generated to FSP 6-21 and 178. Melanoma cells (B16) express both the 6-21 and 178 FS RNA (FIG. 4A), while the breast cancer line, 4T1, only expresses the 178 RNA (FIG. 4C). FIGS. 4A-4C shows ∂-6-21 staining of B16 Melanoma cells (FIG. 4A) and 4T1 breast cancer cells (FIG. 4C) treated. The staining of B16 melanoma cells is mainly on protruding outside of the membrane while the 4T1 breast cancer likely non-specific, background staining as the nucleus also stained. A high titer antibody was used with a 1:100 dilution.

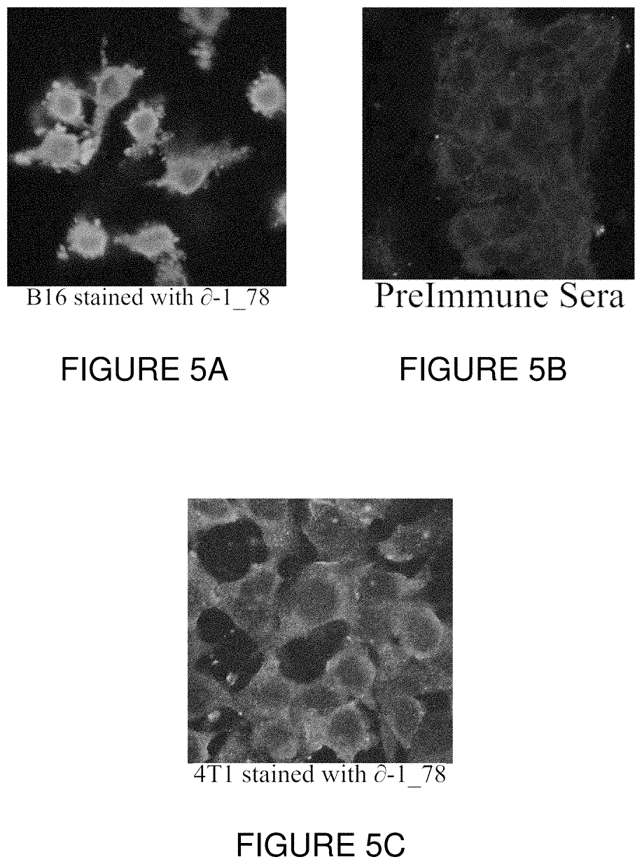

FIGS. 5A-5C show ∂-1-78 staining of B16 Melanoma cells (FIG. 5A) and 4T1 breast cancer cells (FIG. 5C). The staining of B16 melanoma cells and 4T1 cells is mainly on the membrane and protruding outside of the membrane. A high titer antibody was used with a 1:100 dilution.

Throughout this application, various publications are referenced. The disclosures of these publications in their entireties are hereby incorporated by reference into this application in order to more fully describe the state of the art to which this invention pertains. The references disclosed are also individually and specifically incorporated by reference herein for the material contained in them that is discussed in the sentence in which the reference is relied upon.

The present disclosure is not to be limited in scope by the specific embodiments described herein. Indeed, various modifications of the disclosure in addition to those described herein will become apparent to those skilled in the art from the foregoing description and the accompanying figures. Such modifications are intended to fall within the scope of the appended claims.

What is claimed is:

1. A method of producing a therapeutic antibody or a preventative antibody specific for a cancer of interest, comprising:
providing one or more biological samples obtained from one or more subjects identified as having the cancer of interest to a substrate comprising frameshift peptides produced by one or more tumors;
detecting antibodies which bind to the frameshift peptides;
selecting the frameshift peptides that are immunoreactive with the antibodies; and
preparing an antibody composition against the selected frameshift peptides for the cancer of interest.

2. The method of claim 1, wherein the one or more biological samples comprises a set of biological samples obtained from the one or more subjects using antibody reactivity.

3. The method of claim 2, wherein the antibody reactivity is detected using an antibody assay comprises ELISA, radioimmunoassay, Western blot, surface plasmon resonance, immunostaining, immunoprecipitation, mass spectrometry, phage display, ELISPOT, flow cytometry, cytometric bead array, immunohistochemistry, high density array, microarray, delayed-type hypersensitivity (DTH), and combinations thereof.

4. The method of claim 1, wherein the frameshift peptides comprise peptides encoded by a frameshifted mRNA expressed by a cancer cell.

5. The method of claim 4, wherein the frameshifted mRNA is created in a splicing error, a transcription insertion or deletion error, or mis-initiation of translation error.

6. The method of claim 1, wherein the frameshift peptides bind to at least one MHC subtype.

7. The method of claim 1, wherein the frameshift peptides comprise at least one T cell epitope.

8. The method of claim 1, wherein the frameshift peptides comprise at least on B cell epitope.

9. The method of claim 1, wherein the frameshift peptides are presented on the cancer cell surface.

10. The method of claim 1, wherein the frameshift peptides are part of an array or a phage display library.

11. The method of claim 1, wherein the antibodies which bind to frameshift peptides are screened for binding to and/or killing tumor cells.

12. The method of claim 1, wherein the one or more biological samples comprises one or more of blood, plasma, serum, thymus, bone marrow, spleen, lymph node, bronchoalveolar lavage, breast, central nervous system, cerebrospinal fluid, eye, tears, gastrointestinal tract, saliva, feces, urine, heart, kidney, liver, lung, muscle, pancreas, peripheral nervous system, saliva, skin, thyroid, trachea, and tumor.

13. The method of claim 1, wherein the antibody composition further comprises a pharmaceutically acceptable adjuvant or excipient.

14. The method of claim 1, wherein the antibody composition further comprises an immune checkpoint inhibitor.

15. The method of claim 1, further comprising administering an effective amount of the antibody composition to a subject in need thereof to treat the cancer.

16. The method of claim 15, wherein treating the cancer comprises reducing tumor size, inhibiting tumor growth, reducing tumor burden, increasing survival, or increasing cancer-free survival.

17. The method of claim 15, wherein administering the antibody composition elicits an immune response in the subject against the cancer.

18. The method of claim 1, wherein the cancer is acanthoma, acinic cell carcinoma, acoustic neuroma, acral lentiginous melanoma, acrospiroma, acute eosinophilic leukemia, acute lymphoblastic leukemia, acute megakaryoblastic leukemia, acute monocytic leukemia, acute myeloblastic leukemia with maturation, acute myeloid dendritic cell leukemia, acute myeloid leukemia, acute promyelocytic leukemia, adamantinoma, adenocarcinoma, adenoid cystic carcinoma, adenoma, adenomatoid odontogenic tumor, adrenocortical carcinoma, adult T-cell leukemia, aggressive NK-cell leukemia, AIDS-related cancers, AIDS-related lymphoma, alveolar soft part sarcoma, ameloblastic fibroma, anal cancer, anaplastic large cell lymphoma, anaplastic thyroid cancer, angioimmunoblastic T-cell lymphoma, angiomyolipoma, angiosarcoma, appendix cancer, astrocytoma, atypical teratoid rhabdoid tumor, basal cell carcinoma, basal-like carcinoma, B-cell leukemia, B-cell lymphoma, Bellini duct carcinoma, biliary tract cancer, bladder cancer, blastoma, bone cancer, bone tumor, brain stem glioma, brain tumor, breast cancer, Brenner tumor, bronchial tumor, bronchioloalveolar carcinoma, brown tumor, Burkitt's lymphoma, cancer of unknown primary site, carcinoid tumor, carcinoma, carcinoma in situ, carcinoma of the penis, carcinoma of unknown primary site, carcinosarcoma, Castleman's disease, central nervous system embryonal tumor, cerebellar astrocytoma, cerebral astrocytoma, cervical cancer, cholangiocarcinoma, chondroma, chondrosarcoma, chordoma, choriocarcinoma, choroid plexus papilloma, chronic lymphocytic leukemia, chronic monocytic leukemia, chronic myelogenous leukemia, chronic myeloproliferative disorder, chronic neutrophilic leukemia, clear-cell tumor, colon cancer, colorectal cancer, craniopharyngioma, cutaneous T-cell lymphoma, Degos disease, dermatofibrosarcoma protuberans, dermoid cyst, desmoplastic small round cell tumor, diffuse large b cell lymphoma, dysembryoplastic neuroepithelial tumor, embryonal carcinoma, endodermal sinus tumor, endometrial cancer, endometrial uterine cancer, endometrioid tumor, enteropathy-associated T-cell lymphoma, ependymoblastoma, ependymoma, epithelioid sarcoma, erythroleukemia, esophageal cancer, esthesioneuroblastoma, Ewing family of tumor, Ewing family sarcoma, Ewing's sarcoma, extracranial germ cell tumor, extragonadal germ cell tumor, extrahepatic bile duct cancer, extramammary Paget's disease, fallopian tube cancer, fetus in fetu, fibroma, fibrosarcoma, follicular lymphoma, follicular thyroid cancer, gallbladder cancer, gallbladder cancer, ganglioglioma, ganglioneuroma, gastric cancer, gastric lymphoma, gastrointestinal cancer, gastrointestinal carcinoid tumor, gastrointestinal stromal tumor, gastrointestinal stromal tumor, germ cell tumor, germinoma, gestational choriocarcinoma, gestational trophoblastic tumor, giant cell tumor of bone, glioblastoma multiforme, glioma, gliomatosis cerebri, glomus tumor, glucagonoma, gonadoblastoma, granulosa cell tumor, hairy cell leukemia, hairy cell leukemia, head and neck cancer, head and neck cancer, heart cancer, hemangioblastoma, hemangiopericytoma, hemangiosarcoma, hematological malignancy, hepatocellular carcinoma, hepatosplenic T-cell lymphoma, hereditary breast-ovarian cancer syndrome, Hodgkin's lymphoma, hypopharyngeal cancer, hypothalamic glioma, inflammatory breast cancer, intraocular melanoma, islet cell carcinoma, islet cell tumor, juvenile myelomonocytic leukemia, Kaposi's sarcoma, kidney cancer, Klatskin tumor, Krukenberg tumor, laryngeal cancer, laryngeal cancer, lentigo maligna melanoma, leukemia, lip and oral cavity cancer, liposarcoma, lung cancer, luteoma, lymphangioma, lymphangiosarcoma, lymphoepithelioma, lymphoid leukemia, lymphoma, macroglobulinemia, malignant fibrous histiocytoma, malignant fibrous histiocytoma, malignant fibrous histiocytoma of bone, malignant glioma, malignant mesothelioma, malignant peripheral nerve sheath tumor, malignant rhabdoid tumor, malignant triton tumor, MALT lymphoma, mantle cell lymphoma, mast cell leukemia, mediastinal germ cell tumor, mediastinal tumor, medullary thyroid cancer, medulloblastoma, medulloepithelioma, melanoma, meningioma, Merkel cell carcinoma, mesothelioma, metastatic squamous neck cancer with occult primary, metastatic urothelial carcinoma, mixed Mullerian tumor, monocytic leukemia, mouth cancer, mucinous tumor, multiple endocrine neoplasia syndrome, multiple myeloma, mycosis fungoides, myelodysplastic disease, myelodysplastic syndromes, myeloid leukemia, myeloid sarcoma, myeloproliferative disease, myxoma, nasal cavity cancer, nasopharyngeal cancer, nasopharyngeal carcinoma, neoplasm, neurinoma, neuroblastoma, neurofibroma, neuroma, nodular melanoma, non-Hodgkin lymphoma, nonmelanoma skin cancer, non-small cell lung cancer, ocular oncology, oligoastrocytoma, oligodendroglioma, oncocytoma, optic nerve sheath meningioma, oral cancer, oropharyngeal cancer, osteosarcoma, ovarian cancer, ovarian epithelial cancer, ovarian germ cell tumor, ovarian low malignant potential tumor, Paget's disease of the breast, Pancoast tumor, pancreatic cancer, papillary thyroid cancer, papillomatosis, paraganglioma, paranasal sinus cancer, parathyroid cancer, penile cancer, perivascular epithelioid cell tumor, pharyngeal cancer, pheochromocytoma, pineal parenchymal tumor of intermediate differentiation, pineoblastoma, pituicytoma, pituitary adenoma, pituitary tumor, plasma cell neoplasm, pleuropulmonary blastoma, polyembryoma, precursor T-lymphoblastic lymphoma, primary central nervous system lymphoma, primary effusion lymphoma, primary hepatocellular cancer, primary liver cancer, primary peritoneal cancer, primitive neuroectodermal tumor, prostate cancer, pseudomyxoma peritonei, rectal cancer, renal cell carcinoma, respiratory tract carcinoma involving the NUT gene on Chromosome 15, retinoblastoma, rhabdomyoma, rhabdomyosarcoma, Richter's transformation, sacrococcygeal teratoma, salivary gland cancer, sarcoma, Schwannomatosis, sebaceous gland carcinoma, secondary neoplasm, seminoma, serous tumor, Sertoli-Leydig cell tumor, sex cord-stromal tumor, Sezary syndrome, signet ring cell carcinoma, skin cancer, small blue round cell tumor, small cell carcinoma, small cell lung cancer, small cell lymphoma, small intestine cancer, soft tissue sarcoma, somatostatinoma, soot wart, spinal cord tumor, spinal tumor, splenic marginal zone lymphoma, squamous cell carcinoma, stomach cancer, superficial spreading melanoma, supratentorial primitive neuroectodermal tumor, surface epithelial-stromal tumor, synovial sarcoma, T-cell acute lymphoblastic leukemia, T-cell large granular lymphocyte leukemia, T-cell leukemia, T-cell lymphoma, T-cell prolymphocytic leukemia, teratoma, terminal lymphatic cancer, testicular cancer, thecoma, throat cancer, thymic carcinoma, thymoma, thyroid cancer, transitional cell cancer of renal pelvis and ureter, transitional cell carcinoma, urachal cancer, urethral cancer, urogenital neoplasm, uterine sarcoma, uveal melanoma, vaginal cancer, Verner Morrison syndrome, verrucous carcinoma, visual pathway glioma, vulvar cancer, Waldenstrom's macroglobulinemia, Warthin's tumor, or Wilms' tumor.
